SEQUENCE LISTING (33 sequences)

SEQ ID NO: 1 - DNA, 1701 bp, Influenza A virus
atggaagcaaaactactcgtattattctgcatgttcgctgtgctaaaagccgacaccatctgtataggctatcatgcaaacaactctacagacactgtcgacacagtgctagaaaagaatgtaaccgtgactcactcagttaatttgctcgaagacaaccataatggaaaactctgcaagctgaacgggatagccccattacaactaggaaaatgcaacgtagcggggtggctcctcggcaatccggagtgtgacctgctgctcactgcgaattcatggtcctatataatagaaacttcaaattcagaaaatggaacgtgctaccccggggaattcattgattatgaagaattgcgagaaaaactaagttcagtgtcttcatttgagaaatttgagattttcccgaaggcaagctcgtggccaaatcatgagacaaccaaaggtgtcacagctgcatgctcttactctggagctagcagtttttatcgaaatttgctgtggataacaaagaagggaaattcctatcctaaactcagtaaatcatacacgaacaacaagggaaaagaagtacttgtactctggggagtgcatcaccctccaactacaaatgaacaacagaccctctatcagaatactggcgcatatgtctcagtaggatcatcaaagtataaccggaggttcacacctgaaatagcggccagacctaaagtcagagggcaagctggtaggatgaactattattggacactattggatcaaggagacacaataacatttgaagcaactgggaacttaatagcaccatggtatgcttttgcactaaataagggctctgattctggtattataacatcagatgctccggttcatgattgtgacacaaagtgccaaacacctcatggtgccattaacagtagtctccctttccagaacgtgcatcctatcactattggagaatgccctaaatatgttaaaagcaccaaactaaggatggcaacaggcctgagaaacattccttccgttcaatccagaggactctttggagcaattgctggattcattgaaggaggatggactggaatgatagatgggtggtatgggtatcatcatcagaatgagcaaggttctggttatgcagctgatcagaagagcacacaaaatgcgatcgatggaataaccaacaaagtgaactcagtaattgagaaaatgaacactcaattcactgcagtgggtaaggagttcaacaatctggagagaagaattgagaatctgaataaaaaagttgacgatgggtttctggacatttggacgtacaatgcagaattgctcgttttgctcgagaatgaaaggaccttggatttccatgactccaatgtgaggaatctgtatgaaaaggtcaaatcacaattgaggaataatgccaaagaaatcgggaacgggtgcttcgagttctatcacaaatgtgatgacgagtgcatggagagtgtgaaaaacggcacatatgactatcccaagtactcagaggagtccaaactaaacagagaagagatagacggggtaaaattggaatcaatgggggtttaccagattttggcgatctactccacagtcgccagttccctggtcttgttagtctccctgggggcaatcagtttctggatgtgttctaatgggtcattgcaatgcagaatatgcatttag

SEQ ID NO: 2 - DNA, 1773 bp, Influenza A virus
agcaaaagcaggggttataccatagacaatcaaagcaaagacaatggccatcatttatctaatccttctgttcacagcagtgagaggggaccagatatgcgttgggtaccattccaacaattccacagaaaaggttgacacaattctagagagaaatgtcactgtgactcacgctcaggacattcttgagaagactcacaatggaaagttatgcaaactgaacggaatccctccacttgaattaggggattgcagcattgccggatggctccttggaaatccagaatgtgatagacttctaactgtgccagaatggtcatatataatggagaaagaaaatccaaggaatggtttgtgctacccaggcagtttcaatgattatgaagaattgaaacatcttctcagcagagtgacacatttcgagaaagtaaagattctgcccaaagataggtggacacagcatacaacaactggaggttcacgggcttgcgcagtatctggtaatccatcattcttcaggaacatggtctggttgacaaagaaaggatcgaattatccagttgccaaaggatcatacaacaatacaagtggagaacaaatgctgatcatttggggagtacaccaccccaatgatgaaactgaacaaagaacattgtatcagaatgtggggacctatgtgtcagtaggaacatcaacattgaataaaaggtcactcccagaaatagcaacaagacctaaagtgaatggacaaggaggtagaatggaattctcgtggactatcttagatatatgggacaccataaatttcgagagcactggtaatctaattgcaccagaatatggtttcaaaatatccaaacgaggtagttcagggatcatgaaaacagaaggaacacttgaaaactgcgagaccaagtgccaaagtcctttgggagcaataaatacaacgttgccctttcacaatatccacccactgaccattggtgagtgccccaaatatgtaaaatcggaaagattagtcttagcaacaggactaagaaacgtccctcagattgaatcaaggggattgtttggggcaatagctggttttatagaggggggatggcaaggaatggttgatggttggtatgggtaccatcacagcaacgaccagggatccgggtatgcagcagacaaagaatccactcaaaaggcaattgatggaatcaccaacaaggtaaattctgtgattgaaaagatgaacacccaattcgaagctgttggaaaagaattcagtaacttggagaaaagactggagaacttgaataaaaagatggaggacggatttctaggtgtgtggacatacaatgccgagctcctggttctaatggaaaatgagaggacacttgactttcatgattctaatgtcaagaatctatatgataaagtcagaatgcaactgagggacaatgcgaaagagctagggaatggatgttttgaattttatcacaaatgtgatgatgaatgtatgaacagtgtgaagaatgggacatatgattatcccaagtatgaagaggagtctaaactaaacaggaatgaaatcaaaggggtaaaattgagcaatatgggggtttatcaaatccttgccatctatgctacagtagcaggttccctgtcactggcaatcatgatagctgggatctctttatggatgtgctccaacgggtctctgcaatgcagaatctgcatatgattatcagtcattttataattaaaaacacccttgtttctact

SEQ ID NO: 3 - DNA, 1731 bp, Influenza A virus
taattctattaaccatgaagactatcattgctttgagccacattttatgtctggttttcgctcaaaaacttcccggaaatgacaacagcacagcaacgctatgcctgggacatcatgcagtgccaaacggaacgctagtgaaaacaatcacgaatgatcaaattgaagtgactaatgctactgagttggttcagagttcttcaacaggtagaatatgcgacagtcctcaccgaatccttgatggaaaaaactgcacactgatagatgctctattgggagaccctcattgtgatggcttccaaaataaggaatgggacctttttgttgaacgcagcaaagcttacagcaactgttacccttatgatgtgccggattatgcctcccttaggtcactagttgcctcatcaggcaccctggaatttatcaatgaagacttcaattggactggagtcgctcaggatgggggaagctatgcttgcaaaaggggatctgttaacagtttctttagtagattgaattggttgcacaaattagaatacaaatatccagcgctgaacgtgactatgccaaacaatggcaaatttgacaaattgtacatttggggggttcaccacccgagcacggacagtgaccaaaccagcctatatgttcgagcatcagggagagtcacagtctctaccaaaagaagccaacaaactgtaaccccgaatatcgggtctagaccctgggtaaggggtctgtccagtagaataagcatctattggacaatagtaaaaccgggagacatacttttgattaatagcacaggaaatctaattgctcctcggggttacttcaaaatacgaaatgggaaaagctcaataatgaggtcagatgtacccattggcaactgcagttctgaatgcatcactccaaatggaagcattcccaatgacaaaccttttcaaaatgtaaacaggatcacatatggggcctgccccagatatgttaagcaaaacactctgaaattggcaacagggatgcggaatgtaccagagaaacaaactagaggcatattcggcgcaatcgcaggtttcatagaaaatggttgggagggaatggtagacggttggtatggtttcaggcatcaaaattctgagggcacaggacaagcagcagatcttaaaagcactcaagcagcaatcgaccaaatcaacgggaaactgaataggttaatcgagaaaacgaacgagaaattccatcaaatcgaaaaagaattctcagaagtggaagggagaattcaggacctcgagaaatatgttgaagacactaaaatagatctctggtcttacaacgcggagcttcttgttgccctggagaaccaacatacaattgatctaactgactcagaaatgaacaaactgtttgaaaaaacaaggaagcaactgagggaaaatgctgaggacatgggcaatggttgcttcaaaatataccacaaatgtgacaatgcctgcatagggtcaatcagaaatggaacttatgaccatgatgtatacagagacgaagcattaaacaaccggttccagatcaaaggtgttgagctgaagtcaggatacaaagattggatcctatggatttcctttgccatatcatgctttttgctttgtgttgttttgctggggttcatcatgtgggcctgccaaaaaggcaacattaggtgcaacatttgcatttgagtgcattaattaaaaa

SEQ ID NO: 4 - DNA, 1738 bp, Influenza A virus
agcgaaagcaggggaaacaatgctatcaattgtaattttgtttctgctcgtagcagagaattcttctcaaaactatacaggaaaccctgtgatatgcatgggacatcatgctgttgccaatggaaccatggtaaagaccctaaccgatgatcaagtagaagtggtcactgcacaagaactggtagaatcgcagaatctcccggaattatgtccgagtccactgagactagttgatggtcagacttgcgatatcatcaatggagcattggggagcccaggatgtgaccatctgaatggtgcagaatgggatgtgttcatagaaagacccaatgcaatggacacctgctatccattcgatgtgccagattaccaaagcctgagaagcatacttgcgaacaatgggaaattcgaattcattgcagaagaatttcaatggactacagtgaagcaaaatgggaaatctggagcttgcaagagggcaaacgtgaatgatttctttaacaggctaaattggctggtaaagtcagacggaaatgcgtaccctcttcaaaatttgacaaaagtaaataacggtgattatgcaaggctttacatttggggggtccatcacccttcaacagatacagagcaaactaatctatacaagaacaacccgggaagagtcactgtctctaccaagactagccaaacaagtgtagtccctaacattggtagcagaccttgggtgagaggacaaagcggcaggataagtttctactggactatcgtagagcctggagacctaatagtcttcaatacaatagggaatctaattgccccaagaggacattacaagctaaacaatcagaagaagagtacaattctgaacactgcaattcccataggctcatgtgttagcaaatgtcacacagacaaaggctctctctccacgactaaacctttccagaacatctcaagaatagcaatcggggattgccccaaatatgttaaacagggatctttaaaacttgcaacagggatgaggaacatccctgaaaaggcatcaagaggactctttggagcaatagctggattcatagagaatggctggcagggcctaatcgatggttggtatggattcagacatcaaaatgcagaaggaacaggaactgcagcagacctcaaatccacccaggcagccatcgatcagatcaatgggaagttgaaccgtcttatcgaaaagacaaatgagaaataccatcaaattgaaaaagagttcgaacaggttgagggaagaattcaagatttagaaaaatatgttgaggatacaaagattgatttatggtcatataatgcagaactgctagtagcactggaaaaccagcacaccatagatgtgactgattcggagatgaacaaactttttgaaagagtgaggcgtcaactcagagaaaatgctgaagacaaaggaaatggatgtttcgagatatttcacaaatgtgacaacaattgcattgagagcatccggaatggaacttatgatcatgacatttacagaaatgaggcaatcaacaaccgattccaaattcaaggagtcaaattgacccagggatacaaagacatcattctttggatttcattctccatgtcatgctttttgctcgttgcactgcttttagcctttattttgtgggcttgtcaaaacggaaacatcaggtgccagatttgcatttagagaaaaaacacccttgtttctact

SEQ ID NO: 5 - DNA, 1734 bp, Influenza A virus
ccaaactatgaaaatggaaagaatagtgattgccctcgcaataatcagcattgtcaaaggtgaccaaatttgcattggttaccatgcaaacaattcaacagagcaggttgatacaatcatggaaaagaatgtgacggtcacacatgctcagaatatactggaaaaagagcacaatgggaaactttgcagtcttaaaggagtgaggcccctcattctgaaggattgcagtgtagctggatggcttcttggaaacccaatgtgtgatgaattcctgaatgtaccagaatggtcatacatcgtggaaaaagataatccagtcaatggcctgtgctatccaggagacttcagcgactacgaagaactgaagcatttaatgagcagcacaaaccattttgagaaaattcagataatccctaggagttcttggtccaatcatgatgcctcatcaggagtgagttcggcatgcccatacaatggtagatcttccttattcaggaatgtagtgtggttgatcaagaagaataatgcgtacccaacagtaaagaggacctataacaacaccaatgtagaagaccttttaataatatggggaatacaccaccctaatgatgcagctgaacaaacaaaactctaccagaactcgaacacttatgtgtctgtaggaacatcaacactgaaccagagatcaatcccagaaatagccactagacccaaagtgaacggacaaagtggaagaatggaatttttctggacaatactgaagtcgaacgatgcaatcagctttgaaagtaatgggaattttatagctcctgaatatgcgtacaaaattgccaagaaaggagattcagcaatcatgagaagtgaattggagtatggtaactgtgacaccaaatgtcagactccattgggtgctataaattccagtatgcccttccacaatgttcatcctcttaccattggggagtgccccaagtatgtcaaatcggacaaactggtccttgcaacaggactaagaaacgtaccccaaagagaaacaagaggcctatttggtgcaatagcaggattcatagaaggaggatggcaaggaatggttgacgggtggtacggataccatcatagcaatgagcagggaagtggatatgctgcagacaaagaatctacccagaaagcaatcgatgggatcaccaatagagtaaactcaatcattgacaaaatgaacactcaattcgaagccgttgggaaagaattcaacaacctagaaaggagaatagaaaatttgaataagaaaatggaagatgggtttttagatgtatggacttacaatgcagaacttctcgtgctcatggaaaacgaaagaactctggatttccatgattcaaatgtcaagaacctatacgataaggtccgactccagctgagagacaatgcaaaagaattgggcaacggatgcttcgaattctaccacaagtgtgacaatgaatgcatggaaagtgtgagaaatggaacgtatgactatccgcaatattcagaagaatcaagactgaacagagaggaaatagacggagtcaaattggaatcaatgggcacctatcagatattatcaatctactcaacagtggcgagttccctagcactggcaatcatgatagctggtctatctttttggatgtgctccaatggatcattgcagtgcagaatttgcatctagaattgtgagttcagattataattaaa

SEQ ID NO: 6 - DNA, 1706 bp, Influenza A virus
atgattgcaatcattgtaatagtgatactggtaacggccggaaagtcagacaagatctgcatcggataccatgccaacaattcaacaacacaagtggatacgatattggagaaaaatgtaaccgtcacacactctgttgaattgctggaaaatcaaaaggaagaaagattctgtcggattttgaacaaggccccacttgacttaagaggatgcaccatggagggttggatattagggaaccctcaatgtgacctattacttggtgatcagagttggtcatacatagtggaacggcctacagctcgaaatgggatctgctaccctggagttttgaatgaagcagaagaattgaaggcactcattggatcaggagaaagagtagagagatttgaaatgtttcccaaaagcacatggagaggagtagacaccagcaacggagtaacagcagcttgcccttacaatagtggttcttctttctacagaaacctgctatggataataaaaaccacttcagcacaatatccagtaattagtggaacttacaataacactgggaacaagccaatcctctatttctggggtgtacatcatcctcctgataccactgaacaaaacaagttatatggttctggcgatcgatatgttagaatgggaactgaaagcatgaattttgccaagagcccggaaattgcagcaaggcctgcagtgaatggccaaagaggcaggattgattattactggtctgttttacggccaggggaaacattaaatgtggaatctaatgggaatctaatcgctccttggtatgcatacaaatttgacagtaccactagcaagggagcagtattcaagtcagatttgccaatagagagctgcgatgccacatgtcaaactgttgctggagtcctgagaacaaatagaacctttcagaatgtaagccccctgtggataggagagtgtcccaaatacgtgaaaagcgaaagcttgaggcttgcaactgggctaagaaatgtcccaaaggttgataccagagggcttttcggagccatagcaggtttcatcgaagggggatggactggaatggtagatgggtggtatggctatcatcatgagaactctcaaggttcggggtatgcagcagacagggaaagcacccaaaaggccatagacgggattacaaataaggtcaattccattatagacaagatgaacacacaattcgaagctgttgaccatgagttttcaaacttggagaggagaattgacaacctacacaaaagaatggaggatgggtttctggatgtttggacatacaatgctgaactgttggttctcctcgaggacgaaaggacactggacatgcatgacgctaacgtgaagaatctgtatgaaaaggtcaaatcacaactacgagacaatgctaatgatatgggaaatgggtgctttgaattttggcataagtgtgatgatgaatgcatggagtctgtcaaaaatggaacttatgattatccaaaataccaggatgaaagtaagttaaacagacaggaaatagaatcagtgaagctggagaatcttggtgtgtatcaaattcttgccatctatagtacggtatcgagcggtctagtattggtggggctgatcatagcaatgggcctttggatgtgttcgaatggttcaatgcaatgcaggatatgtatataattaag

SEQ ID NO: 7 - DNA, 1686 bp, Influenza A virus
atgaacactcaaattctggcattcattgcctgtatgctggttggagttaaaggagacaaaatatgtcttgggcaccatgctgtggcaaatgggacaaaagtgaacacactaacagagaggggaattgaagtcgtgaatgccacagagacggtggagaatgcgaatattaagaaaatatgcactcaagagaaaaggccaacagatctgggacaatgtggacttctagggaccttaataggacctccccaatgtgaccgattcctggagtttgattctgatttaatcattgagcgaagggaaggaaccgatgtgtgctatcccggaaaattcacaaatgaagaatcactgaggcagatccttcgagggtcaggaggaattgataaggagtcaataggtttcacttatagtggaataagaaccaatggagtgacaagtgcctgcagaagattaggttcttcttccttctatgcagagatgaagtggttactgtcgaattcagacaatgcggcatttccccagatgaccaaatcgtatagaaatcccaggaacaagccagctctgataatttggggaattcatcactctgcatcggctaatgagcagaccaaactctatggaagtggaaacaaattgataacagtaggaagctcgaaataccagcaatcattcactccaagtccgggagcacggccacaagtgaatggacaaacagggaggatcgatttccactggctgctccttgatcccaatgacacggtgaccttcaccttcaatggagcattcatagcccctgacagggcaagtttctttagaggggaatcgctaggaatccagagtgatgttcctttggattctagttgtgggggagactgctttcacagcgggggtacgatagtaagttccctgccattccaaaacatcaaccctagaactgtggggaaatgccctcgatatgtcaaacagacaagcctccttttggctacaggaatgagaaatgtcccagagaatcccaagaccaggggtctctttggagcaattgctggattcatagagaatggatgggagggtctcattgatggatggtatggtttcaaacatcaaaatgcgcaaggagaaggaactgcagctgattataaaagcacccaatctgcaatagatcagatcacaggcaaattgaatcgtttgatcgacaaaacaaatcagcagtttgagctgatagacaatgagttcaatgaaatagaacaacaaatcggaaatgtcattaattggacacgagattcaatgactgaagtgtggtcgtacaatgctgaactgttggtggcaatggaaaatcagcataccatagatcttgcggactcagaaatgaacaaactttatgagcgtgtcaggaaacaactaagggagaatgctgaagaagatggaactggatgttttgagatattccataagtgtgatgatcagtgtatggagagcataaggaacaacacttatgaccatactcaatacagaacagagtcattacagaatagaatacagatagacccagtgaaattgagtagtgggtacaaagacataatcttatggtttagcttcggggcatcatgtttccttcttctagccattgcaatgggattgatcttcatttgcataaagaatggaaacatgcggtgcactatttgtatatag

SEQ ID NO: 8 - DNA, 1672 bp, Influenza A virus
ctcttggtgagcacaaatgcatacgataggatatgcattggttaccaatcaaacaactccacagacacagtgaacacccttatagaacagaatgtgccagtcactcaaacaatggagctcgtggaaacggagaaacatcccgcttattgcaacactgatttaggatcaccattggaactacgagactgcaaaattgaagcagtaatctatggaaatcccaagtgtgacatccatctaaaggaccaagattggtcatacatagtggagaggcccagtgcaccagagggaatgtgttaccctggatctgtggaaaatctagaagaactgagattcgtcttctccaatgctgcatcctacaagagaataagactatttgactattccaaatggaatgtgaccagctctgggaccagcaaggcatgcaatgcatcaacaggtggccaatcattctataggagcatcaactggttgaccaagaaaaaaccagacacttacgatttcaatgaagggagttatgttaacaatgaggatggagacatcattttcttatgggggattcatcatccacctgatgcaaaagagcaaacgacactgtacaagaatgcaaacaccttgagtagtgttactaccaacaccataaatagaagttttcaacccaatattggtccaaggccattagtcagaggacaacaagggagaatggattactactggggcattctgaaaaggggagagactctgaaaattaggactaatggaaatttaattgcacctgaatttggatatttacttaagggtgagagccatggcagaataattcaaaatgaggacatccccattggaaactgccacacaaaatgccagacatatgcaggagcaatcaatagcagcaaaccctttcagaatgccagtagacattatatgggggagtgtcccaaatatgtaaaaaaggcaagcttgcgacttgcagtgggacttagaaacacaccttctattgaacccaaagggctatttggagccattgctggtttcatcgagggaggatggtctgggatgattgatggatggtatggatttcaccacagcaactcagagggaacaggaatggcagctgaccagaagtcaacacaggaagccatcgataagatcaccaataaagtcaacaatatagttgataagatgaacagggagtttgaagttgtgaatcatgagttctctgaagttgaaaaaaggataaacatgataaatgataaaatagatgaccaaattgaagatctttgggcttacaatgcagaactccttgtgcttctagaaaaccagaagacactagacgaacatgactccaatgtcaaaaatcttttcgatgaggtgaaaagaagattgtcggccaatgcaatagatgctgggaatggttgctttgacatactccacaagtgcaacaatgagtgtatggaaactataaagaatgggacttacgaccataaggagtatgaagaggaggctaaattagaaaggagcaaaataaatggggtgaaactggaagagaacaccacttacaaaattcttagcatttacagtacagtggcggccagtctctgcttggcaatcctgattgctggaggtttaatcctgggtatgcaaaatggatcttgtagatgcatgttctgtattt

SEQ ID NO: 9 - DNA, 1683 bp, Influenza A virus
atggaaacagtattgctaataactatactactagtagtaacagtaagcaatgcagataaaatctgcatcggctaccaatcaacaaactccacagaaactgtagacacgctaacagaaaacaatgtccctgtgacacatgccaaagaattgctccacacagagcacaatgggatgctgtgtgcaacaaatctgggacatcctctcattctagacacctgtaccattgaaggactaatctatggcaacccttcttgtgatctactgttgggaggaagagaatggtcctacatcgttgaaagaccatcggctgttaatggattgtgttaccccgggaatgtagaaaacctagaagaactaaggtcactttttagttctgctagttcttaccaaagaatccagatctttccagacacaatctggaatgtgtcttacagtgggacaagcaaagcatgttcagattcattctacagaaacatgagatggttgactcaaaagaacaacgcataccctattcaagacgcccaatacacaaataatcgagggaagaacattcttttcatgtggggcataaatcacccacccaccgatactacgcagacaaatctgtacacaaggactgacacaacaacaagtgtggcaacagaagatataaataggaccttcaaaccattgatagggccacggcctcttgtcaatggtcagcagggaagaattgattattattggtcggtattgaaaccaggtcaaacactgcgagtaaggtccaatgggaatctaattgctccattgtatggacacatcctttcaggagagagccacggaagaatcctgaagactgatttaaaaagtggtaactgtgcagtgcaatgtcagacagaaagaggtggcttaaatacaacattgccattccaaaatgtaagtaagtatgcatttggaaactgcccgaaatatgttggagtaaagagtctcaaactggcagttggtctgaggaatgtgcctgctagatcaagtagaggactattcggggccatagctggattcatagagggaggttggtcagggctagtcgctggttggtatgggttccagcattcaaatgatcaaggggttggtatggcagcagatagagactcaactcaaaaggcaattgacaaaataacatccaaagtgaataatatagtcgataaaatgaacaaacagtatgaaattattgatcatgaattcagcgaggttgaaactagaatcaacatgatcaataataagattgatgaccaaattcaagacatatgggcatataacgcagaattgctagtgctgcttgaaaaccagaaaacactcgatgagcatgatgcgaatgtaaacaatctatataataaagtgaagagggcattgggttccaatgcagtggaagatgggaaaggatgtttcgagctataccataaatgtgatgaccagtgcatggagacaattcggaacgggacctacaacaggaggaagtataaagaggaatcaagattagaaagacagaaaatagaaggggtcaagctggaatctgaaggaacttacaaaatcctcaccatttattcgactgtcgcctcatctcttgtgattgcaatggggtttgctgccttcttgttctgggccatgtccaatggatcttgcagatgcaacatttgtatataa

SEQ ID NO: 10 - DNA, 1760 bp, Influenza A virus
atgtacaaacttgtagcagtaattgcgctccttggagcagtgagaggtcttgacaaaatctgtctaggacatcatgcagtagccaatggaaccatcgtgaagacccttacaaatgaacaggaagaggtgaccaatgctactgaaacagtggagagcacaagtctaaacaggttatgtatgaagggaagaagctataaggatctgggcaattgccatccaataggaatgttgataggaacaccagcttgtgatctgcatcttactgggacatgggacactctcattgagcgagagaatgccattgcctactgttaccctggagcgaccgtaaatgaagaagcactgaggcagaaaataatggagagtgggggaatcagcaaaataagcactggcttcacttatgggtcttccatcaactcagccggaaccactaaagcatgcatgagaaatggaggaaatagcttttatgcagagctcaaatggctagtatcaaagagtaagggacaaaacttccctcagaccacgaacacttatagaaacacggacacagctgaacatctcataatatggggaatccatcacccttccagcactcaggagaagaatgatctatatgggacacaatcactatctatatcagttgggagttccacatatcagaacaattttgttccagttgttggggcaagacctcaggtcaatggacaaagcgggcgaattgatttccactggacattagtacaaccaggcgataatatcactttctcacacaatgggggtctgatagcaccgagtcgagttagcaaactagttggaagaggattgggagttcaatcagaagcatcaatagacaatggttgtgaatctaaatgtttttggagaggaggctccataaatacaaagctccctttccaaaatttgtcaccaaggacagtgggtcagtgtcccaaatacgtgaataagaagagtttgatgcttgcaacaggaatgagaaatgtaccagaaataatgcaagggagaggtctatttggtgcaatagcaggatttatagaaaatggatgggaaggaatggtggatggttggtatggtttcagacaccaaaatgctcaggggacaggcctagccgccgattacaagagtactcaggcagctatcgaccaaatcactgggaagctgaacaggctgattgagaagaccaacactgagtttgagtcaatagagtctgagttcagtgagattgaacatcagattggtaacgtcattaattggaccaaggattcaataaccgacatctggacttatcaggaagaactattggtagcaatggagaaccagcatacaattgacatggctgattcagagatgctaaatttgtatgagagagtgaggaaacaactcaggcagaatgcagaagaagatgggaaggggtgttttgagatatatcatacttgtgacgattcgtgcatggagagcataaggaacaacacctatgaccattcacaatacagagaggaagctcttttgaatagactgaacatcaattcagtaaaactttcttcgggttataaagacatcatcctttggtttagcttcggggcatcatgctttgttcttctggccgttgtcatgggtcttgttttcttctgtctgaaaaatggaaacatgcgacgcacaatctgtatttagttaaaaacacccaaacccccttgggaccctgagaggcacccccaagaattagtaaaaaaccccccaaaattttt

SEQ ID NO: 11 - DNA, 1727 bp, Influenza A virus
aatatcaagaaatcaagatgaagaaagtactgctctttgcaacaatcatcatctgcatccaagcagacgagatctgcattgggtacctgagcaacaactcaacagaaaaagtggacacaataattgagagtaatgtcacggttactagctcagttgaactggttgaaaatgagcacactggatctttctgctcaatcgacgggaaagcaccgataagtcttggtgattgctcctttgctggatggatccttggaaacccaatgtgtgatgatttgattgagaaaacatcatggtcctacatagtggagaaaccaaatcccgtcaatggaatatgttacccgggtactctagaaaatgaggaggaattgagactgaagtttagtggagtcctcgaattcagtaaatttgaagccctcacctcaaatggatggggagcagtaaattctggtgctggtgttaccgcagcctgcaaatttggaagcagtaactcttttttcaggaacatggtatggttgatccaccaatcagggacatatcctgttatacggaggacattcaacaacaccaaaggaagagatgtattaatggtatggggagttcaccaccccgcaaccctaaaggaacaccaagacttgtacaaaaaagacagttcttatgtagcagtaggttcagagagttacaacaggaggttcactcctgagatcagtacaaggcctaaagtaaatggtcaggctgggagaatgacattctactggaccatagtgaaaccgggagaagcaataacatttgagtcaaatggtgcatttctcgctcctcggtatgcttttgagttagtatccttggggaatgggaaattgttcaggagtgatttgaatattgaatcttgctcaactaaatgccagtccgaaattggagggatcaacactaataggagcttccacaatgtccataggaacacaataggagactgccccaaatatgtgaatgttaaatctctaaagcttgctaccgggctcagaaatgtccctgcgattgctacaagaggcctgtttggtgcaatagctggtttcatagaaggtggttggccaggtttaatcaatggttggtatgggttccaacacagaaatgaagaaggtacagggattgctgcagacaaagaatcaacccagaaagcaatagaccagataacatctaaagtcaacaacattgttgatcggatgaatacgaactttgagtctgttcaacatgaatccagtgaaattgaagagagaataaaccaattgtcaaaacatgttgatgattctgttattgacatatggtcatacaacgcacagcttcttgttctattggaaaatgaaaaaacattagatctccatgattctaatgttcgaaacctccatgaaaaggtcagacgaatgctgaaggacaatgctaaagatgaagggaatggttgttttactttctatcacaagtgtgataatgaatgtattgaaaaagtcaggaatggaacatatgaccacaaggaatttgaagaggagtccaaattgaatagacaagaaattgagggagtgaaactggattccaatggcaacgtttacaaaatactgtcaatttacagctgcattgcaagcagtcttgtgttggcagcaatcattatgggattcatcttctgggcgtgtagcaatgggtcatgtagatgtaccatttgcatttagaattgcaataaa

SEQ ID NO: 12 - DNA, 1712 bp, Influenza A virus
ggtcacaatggaaaaattcatcattttgagcactgtcctggcagtaaactttgcatatgacaaaatttgcattggatatcagacaaacaactcgactgagacagtaaacacactaatcgagcaaaacgttccggtgacacaggtggaagaactcgtgcatggtggaatcgatccggtcctatgtggaacggagctagggtcaccattagtgcttgatgattgttcactcgaaggtctcattctaggcaatcccaaatgtgatctttatctgaatggcagggaatggtcatacatagtagagaggcccaaagaaatggaaggagtttgctacccaggatcaattaaaaatcaagaggagttgagatctctgttttcttccatcaagaagtatgaaagagtgaagatgtttgatttcaccaaatggaatgtcacgtacactggaaccagcaaggcctgcaacaatacatcaaaccaaggctcattctataggagcatgagatggttaaccttaaaatcaggacaattcccagtccaaacagatgagtacaagaacaccagagattcggacattgttttcacctgggccatccatcacccaccaacatctgatgaacagataaaattatacaaaaatccggataccctctcctcagtcaccactgatgaaatcaataggagtttcaagcctaacataggaccaaggccattagtgagaggacagcaggggagaatggattactactgggctgtcctcaaacctggacaaactgtcaaaatacagaccaatggtaatcttattgcacctgaatatggtcacttaatcacagggaaatcacatggcaggatactcaagaataacttgcccataggacagtgtgtgactgaatgccaattgaatgaaggggtgatgaatacaagtaaacctttccagaacactagtaagcactatattgggaaatgtcccaaatacataccatcgggaagcctgaaattggcgatagggctcagaaatgttccacaagttcagaacaggggactatttggagcaattgcaggtttcatagagggcggatggccaggactagtggctgggtggtatggatttcagcatcaaaatgcagaggggacaggcatagccgcagacagagacagcactcagaaggcaatagacaatatgcaaaacaaactcaacaatgtcattgacaaaatgaacaaacaatttgaagtggtgaatcatgagttttcagaagtggaaagcagaataaacatgattaattccaaaattgatgatcaaataactgacatatgggcatacaatgctgaactgcttgttctattggaaaatcagaagacattagatgagcatgatgctaatgtaagaaacttacacgatagagtcagaagagtcctaagagaaaatgcaattgatacaggagatggttgctttgaaattctgcataaatgtgacaacaattgcatggacacaatcagaaatgggacatacaatcacaaggagtatgaggaagaaagcaaaattgaacgacagaaaattaatggtgtcaaacttgaggagaattctacatataaaattctgagcatctacagcagtgttgcctcaagcttagttctactgctcatgattattgggggtttcattttcggatgtcaaaatggaaatgttcgttgtactttctgtatttaattaaaaacac

SEQ ID NO: 13 - DNA, 1765 bp, Influenza A virus
agcaaaagcagggaaaatttcaacaaactgacacaagagaaatggacatccaagcagtcgcactcttgatattaaccagcacatgcgtacaggctgatagaatctgtgtgggatacttaagcaccaactcatcagaaaaagttaacacactgttagagaacgatgtcccggttacaagttctgtcgatctggttgagactagccacacaggaacatattgttctttggatggaatcagtccagtgcatttgggagactgtagcttcgagggttggattgtagggaaccctgcctgtgccagcaacatggggatcagagaatggtcatacttgattgaagatccctctgcacctcatggactgtgctacccaggagagttagacaacaatggagaattgaggcacctgttcagtggaatcagatctttcagtagaacagagttgatcgcacctacttcttggggggcagtgaatgatggagtatcatcggcctgccaggatcgaggggccagcagcttttatcgaaatttggtatggtttgtggagagaggcaagcaatatccagtgatccgcgggacctacaacaatacaactggcagagatgttttggtcatgtgggggatacatcatcctgtttccacagacgaggcgagaaaactatatgtcaacaatgacccatatacattggtatctaccagttcatggagtagaaaatacaacttagaaattggaatccggcctggatacaatggccaaaagagttggatgaagatttactggtatttgatgcacccaggggaatcaatcagtttcgaaagcaatggaggattgttggcccccagatatggttatattattgaggaatatggaaaagggcgaattttccaaagtcgcattcgcattgctaaatgcaatactaagtgtcagacatctgttggtgggataaataccaacaaaacatttcaaaacatagagagaaatgcacttggggattgcccaaaatacataaaatctgggcagctcaagttggccaccggacttagaaatgtacctgccatatcaaacagaggattatttggagctattgcaggcttcatagaaggtggttggccaggattaataaatggttggtatggattccaacatcagaacgaacaaggagtgggaatggctgcagacaaagagtcaacacaaaaggctattgatcaaataacaaccaagataaacaatatcattgaaaaaatgaatggaaattatgactccatacgaggtgaattcaatcaagtggaacaaaggataaatatgcttgcagacagaatagatgacgctgtaactgatgtatggtcatacaatgcaaagcttcttgtgttgctagagaacgataaaactctagacatgcatgatgccaatgttagaaacctgcatgaccaagtacgtagagcactaaagaccaatgcaattgatgaggggaatggatgctttgaactcctccataaatgcaatgactcttgcatggagacaataagaaatgggacgtacaatcacacagaatatgaagaagaatcaaaattgaagagacaggaaatagaaggtataaagctgaagtcagacgataatgtttacaaagcattatcgatttacagctgcattgcaagcagtattgtaatggtaggactcatacttgcattcatcatgtggacatgcaacagtggaaactgccggttcaatatctgtatataagtagaaaaaatacccttgtttctact

SEQ ID NO: 14 - DNA, 1749 bp, Influenza A virus
agcaaaagcaggggaaaatgattgcactcatattggttgcactggctctgagccacactgcttattctcagatcacaaatgggacaacaggaaaccccattatatgcttggggcatcatgcagtggaaaacggcacatctgttaaaacactaacagacaatcacgtagaagttgtgtcagctaaagaattagttgagacgaaccacactgatgaactgtgcccaagccccttgaagcttgtcgacgggcaagactgccacctcatcaatggtgcattggggagtccaggctgtgaccgtttgcaggacaccacttgggatgtcttcattgaaaggcccactgcagtagacacatgttatccattcgacgtcccagattaccagagtctcagaagcatcctagcaagcagtgggagtttggagttcatcgccgaacaattcacctggaatggtgtcaaagttgacggatcaagcagtgcttgtttgaggggcggtcgcaacagcttcttctcccgactaaactggctaaccaaagcaacaaatggaaactatggacctattaacgtcactaaagaaaatacgggctcttatgtcaggctctatctctggggagtgcatcacccatcaagcgataatgagcaaacggatctctacaaggtggcaacagggagagtaacagtatctacccgctcggaccaaatcagtattgttcccaatataggaagtagaccgagggtaaggaatcagagcggcaggataagcatctactggaccctagtaaacccaggggactccatcattttcaacagtattgggaatttgattgcaccaagaggccactacaaaataagcaaatctactaagagcacagtgcttaaaagtgacaaaaggattgggtcatgcacaagcccttgcttaactgataaaggttcgatccaaagtgacaaaccttttcagaatgtatcaaggattgctataggaaactgcccgaaatatgtaaagcaagggtccctgatgttagcaactggaatgcgcaacatccctggcaaacaggcaaagggcttatttggggcaattgctggattcattgaaaatggttggcaaggcctgattgatgggtggtatggattcaggcaccaaaatgctgaaggaacaggaactgctgcagacctgaagtcaactcaggcagccattgatcagataaatggcaagctgaacagattgatagagaagacaaatgaaaaatatcaccaaatagaaaaggaattcgaacaggtggaaggaagaatacaagaccttgagaagtacgttgaggacactaagattgatttgtggtcatacaatgctgaattgctagtagcactagagaatcagcacacaatagatgtcacagactccgaaatgaacaagctttttgaaagagtaagaaggcaattaagagagaatgcagaagatcaaggcaacggttgtttcgagatattccatcagtgtgacaacaattgtatagaaagcattagaaacggaacttatgaccacaacatctacagggatgaagccatcaacaatcgaatcaaaataaatcctgtcactttgacgatggggtacaaggacataatcctgtggatttctttctccatgtcatgctttgtcttcgtggcactgattctgggatttgttctatgggcttgtcaaaacgggaatatccgatgccaaatctgtatataaagaaaaaacacccttgtttctactc

SEQ ID NO: 15 - DNA, 1762 bp, Influenza A virus
agcaaaagcaggggatacaaaatgaacactcaaatcattgtcattctagtcctcggactgtcaatggtgaaatctgacaagatttgtctcgggcaccatgccgtagcaaatgggacaaaagtcaacacactaactgagagaggagtggaagtggtcaatgccacggagacagtggagattaccggaatagataaagtgtgcacaaaagggaagaaagcagtggacctgggatcttgtggaatactgggaactatcattgggcctccacaatgtgatcttcatcttgaattcaaagctgatctgataatagaaagaagaaattcaagtgacatctgttacccaggaagattcactaatgaggaagcactgagacaaataatcagagaatctggaggaattgacaaagagtcaatgggctttagatattcaggaataagaacagacggggcaaccagtgcgtgtaagagaacagtgtcctctttctactcagaaatgaaatggctttcatccagcatgaataaccaggtgttcccacaactgaatcagacatacaggaacaccagaaaagaaccagccctaattgtctggggagtacatcattcaagttccttggatgagcaaaataagctatatggaactgggaacaagctgataacagtaggaagctcaaagtaccaacaatcgttttcaccaagtccaggggccaggcccaaagtgaatggtcaggccgggaggatcgactttcattggatgctattggacccaggggatacagtcacttttaccttcaatggtgcattcatagccccagatagagccacctttctccgctctaatgccccttcaggaattgagtacaatgggaagtcactgggaatacagagtgatgcacaaatcgatgaatcatgtgaaggggaatgcttctacagtggagggacaataaacagccctttaccatttcaaaacatcgatagtagggctgtcggaaagtgccccagatatgtgaagcaatcaagcttgccgctggccttaggaatgaaaaatgtaccagagaaaatacgtactaggggactgttcggtgcaattgcaggattcatcgaaaatggatgggaagggctcattgatggatggtatggatttaggcatcagaatgcacaagggcagggaacagctgctgactacaagagtactcaggctgcaattgaccagataacagggaaacttaataggttaattgaaaaaaccaacaaacagtttgaactcatagacaatgagttcactgaagtggagcagcagataggcaatgtaataaactggacaagggactccttgactgagatctggtcatacaatgccgaactgctagtagcaatggagaatcagcatacaattgaccttgcagattctgaaatgaacaaactctatgagagagtgagaagacagctaagggagaatgccgaggaggatggaactggatgttttgagattttccaccgatgtgacgatcaatgtatggagagcatacggaataatacttacaatcacactgaatatcgacaggaagccttacaaaataggataatgatcaatccggtaaagcttagtagtgggtacaaagatgtgatactatggtttagcttcggggcatcatgtgtaatgcttctagccattgctatgggtcttattttcatgtgtgtgaaaaacgggaatctgcggtgcactatctgtatataattatttgaaaaacacccttgtttctact

SEQ ID NO: 16 - DNA, 1718 bp, Influenza A virus
atgatgattaaagtgctctactttctcatcatagtgcttggcaggtattcaaaagcagacaaaatatgcataggatacctaagcaataattcttcagatacagtggacacactaacagagaacggagttcctgtgaccagctcagttgatctcgttgaaacaaatcatactggaacatactgctcattgaatggtatcagtccgattcatcttggtgactgcagctttgagggatggattgtaggaaacccctcctgtgccaccaacatcaacatcagagagtggtcatatttaattgaagaccctaatgcccccaacaaactctgctacccaggggagttagacaacaatggagaactacgacacctcttcagcggagtgaactcttttagcagaacagaactgataaatcccagcaaatggggaaatgttctggatggagtcactgcttcttgcctcgatagaggggcaagcagtttttacaggaacttggtctggctagtgaagcaaaaaattggagaatatcctgttgtaaaaggggaatacaacaacacaacgggcagagatgttctagtactttgggggattcaccatccggatacagaaacaacagccacaaacctatatgtcaacaaaaacccctacacactagtatcaacaaaggagtggagcaaacggtatgaacttgaaattggaaccagaataggcgatggacagagaagttggatgaaactatattggcatctcatgcacccgggagaaaggataatgtttgagagtaatgggggccttatagcacccagatatggatacatcattgagaaatacggaacgggacgaattttccaaagtggagtgagaatggccaaatgcaacacaaagtgtcaaacgtcattaggtgggataaacactaacaaaactttccagaacatagaaagaaatgcccttggggactgcccaaagtacataaagtctggacaactgaagcttgcaactggactgagaaatgttcccattcccattggtgaaagaggtctgtttggtgcaattgcaggcttcatagaaggagggtggcctggtctaattaatggatggtatggtttccaacatcagaacgaacaggggacgggcattgctgcggataaagcttccactcagaaagcaataaatgaaataacaacaaagattaacaacataatagagaagatgaacggaaactatgattcgataagaggggaattcaatcaagtagagaagaggattaacatgctcgccgatcgagttgatgatgcagtaactgatatatggtcgtacaatgctaaacttcttgtactaattgaaaatgatagaacgctggacttgcatgacgcaaatgtcagaaacttacatgatcaggtcaagagggcattgaaaagtaatgccattgatgaaggagatggttgcttcaatcttcttcacaaatgcaatgactcatgcatggaaaccattagaaatgggacctacaatcatgaagattacagggaagaatcacaactgaagaggcaggagattgaggggataaaattaaagactgaagacaatgtttataaagtactgtcaatttatagctgcattgcaagcagtattgtgctggtaggtctcatacttgcatttataatgtgggcatgcagcaatgggagttgccggtttaatgtttgtatataatcggaaaaaacacc

SEQ ID NO: 17 - DNA, 1793 bp, Influenza B virus
caaaatgaaggcaataattgtactactcatggtagtaacatccaatgcagatcgaatctgcactgggataacatcgtcaaactcaccacatgtcgtcaaaactgctactcaaggggaggtcaatgtgactggtgtaataccactgacaacaacacccaccaaatctcattttgcaaatctcaaaggaacagaaaccagagggaaactatgcccaaaatgcctcaactgcacagatctggacgtagccttgggcagaccaaaatgcacggggaaaataccctcggcaagagtttcaatactccatgaagtaagacctgttacatctgggtgctttcctataatgcacgacagaacaaaaattagacagctgcccaaccttctccgaggatacgaacatatcaggttatcaactaataacgttatcaatgcagaaaatgcaccaggaggaccctacaaaattggaacctcagggtcttgccctaacgttaccaatggaaacggatttttcgcaacaatggcttgggccgtcccaaaaaacgacaaaaacaaaacagcaacaaatccattaacaatagaagtaccatacatttgtacagaaggagaagaccaaattaccgtttgggggttccactctgataacgagatccaaatggcaaagctctatggggactcaaggccccagaagttcacctcatctgccaacggagtgaccacacattacgtttcacagattggtggcttcccaaatcaaacagaagacggaggactaccacaaagtggtagaattgttgttgattacatggtgcaaaaatctgggaaaacaggaacaattacctatcaaagaggtattttattgcctcaaaaggtgtggtgcgcaagtggcaggagcaaggtaataaaaggatccttgcctttaattggagaagcagattgcctccacgaaaaatacggtggattaaacaaaagcaagccttactacacaggggaacatgcaaaggccataggaaattgcccaatatgggtgaagacacccttgaagttggccaatgggaccaaatatagacctcctgcaaagctattaaaggaaaggggtttcttcggagctattgctggtttcttagaaggaggatgggaaggaatgattgcaggttggcacggatacacgtcccatggggcacatggagtagcggtggcagcagaccttaagagcacccaagaggccataaacaagataacaaaaaatctcaactctttgagtgagctggaagtaaagaatcttcaaagactaagcggtgccatggatgaactccacaacgaaatactagaactagacgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgtaaccagacctgtctcgacagaatagctgctggtacctttgatgcaggagaattttctctccccacctttgattcactgaatattactgctgcatctttaaatgacgatggattggataatcatactatactgctttactactcaactgctgcctccagtttggctgtaacactgatgatagctatctttgttgtttatatggtctccagagacaatgtttcttgctccatctgtctataagggaagttaagccctgtattttcctttattg

SEQ ID NO: 18 - DNA, 3417 bp, Artificial Sequence
  Chimeric sequences
atgaagaccatcattgctttgagctacattttctgtctggctctcggcgccagcacccagagccccagcgtgttccccctgacccgctgctgcaagaacatccccagcaacgccaccagcgtgaccctgggctgcctggccaccggctacttccccgagcccgtgatggtgacctgggacaccggcagcctgaacggcaccaccatgaccctgcccgccaccaccctgaccctgagcggccactacgccaccatcagcctgctgaccgtgagcggcgcctgggccaagcagatgttcacctgccgcgtggcccacacccccagcagcaccgactgggtggacaacaagaccttcagcgtgtgcagccgcgacttcaccccccccaccgtgaagatcctgcagagcagctgcgacggcggcggccacttcccccccaccatccagctgctgtgcctggtgagcggctacacccccggcaccatcaacatcacctggctggaggacggccaggtgatggacgtggacctgagcaccgccagcaccacccaggagggcgagctggccagcacccagagcgagctgaccctgagccagaagcactggctgagcgaccgcacctacacctgccaggtgacctaccagggccacaccttcgaggacagcaccaagaagtgcgccgacagcaacccccgcggcgtgagcgcctacctgagccgccccagccccttcgacctgttcatccgcaagagccccaccatcacctgcctggtggtggacctggcccccagcaagggcaccgtgaacctgacctggagccgcgccagcggcaagcccgtgaaccacagcacccgcaaggaggagaagcagcgcaacggcaccctgaccgtgaccagcaccctgcccgtgggcacccgcgactggatcgagggcgagacctaccagtgccgcgtgacccacccccacctgccccgcgccctgatgcgcagcaccaccaagaccagcggcccccgcgccgcccccgaggtgtacgccttcgccacccccgagtggcccggcagccgcgacaagcgcaccctggcctgcctgatccagaacttcatgcccgaggacatcagcgtgcagtggctgcacaacgaggtgcagctgcccgacgcccgccacagcaccacccagccccgcaagaccaagggcagcggcttcttcgtgttcagccgcctggaggtgacccgcgccgagtgggagcagaaggacgagttcatctgccgcgccgtgcacgaggccgccagccccagccagaccgtgcagcgcgccgtgagcgtgaaccccggcaagggagcagcttcttcagcagctggtggagcagcttcttcagcagctggtggagcagcttcttcagcagctggtggagcagcttcttcagcagctggtgccagcacccagagccccagcgtgttccccctgacccgctgctgcaagaacatccccagcaacgccaccagcgtgaccctgggctgcctggccaccggctacttccccgagcccgtgatggtgacctgggacaccggcagcctgaacggcaccaccatgaccctgcccgccaccaccctgaccctgagcggccactacgccaccatcagcctgctgaccgtgagcggcgcctgggccaagcagatgttcacctgccgcgtggcccacacccccagcagcaccgactgggtggacaacaagaccttcagcgtgtgcagccgcgacttcaccccccccaccgtgaagatcctgcagagcagctgcgacggcggcggccacttcccccccaccatccagctgctgtgcctggtgagcggctacacccccggcaccatcaacatcacctggctggaggacggccaggtgatggacgtggacctgagcaccgccagcaccacccaggagggcgagctggccagcacccagagcgagctgaccctgagccagaagcactggctgagcgaccgcacctacacctgccaggtgacctaccagggccacaccttcgaggacagcaccaagaagtgcgccgacagcaacccccgcggcgtgagcgcctacctgagccgccccagccccttcgacctgttcatccgcaagagccccaccatcacctgcctggtggtggacctggcccccagcaagggcaccgtgaacctgacctggagccgcgccagcggcaagcccgtgaaccacagcacccgcaaggaggagaagcagcgcaacggcaccctgaccgtgaccagcaccctgcccgtgggcacccgcgactggatcgagggcgagacctaccagtgccgcgtgacccacccccacctgccccgcgccctgatgcgcagcaccaccaagaccagcggcccccgcgccgcccccgaggtgtacgccttcgccacccccgagtggcccggcagccgcgacaagcgcaccctggcctgcctgatccagaacttcatgcccgaggacatcagcgtgcagtggctgcacaacgaggtgcagctgcccgacgcccgccacagcaccacccagccccgcaagaccaagggcagcggcttcttcgtgttcagccgcctggaggtgacccgcgccgagtgggagcagaaggacgagttcatctgccgcgccgtgcacgaggccgccagccccagccagaccgtgcagcgcgccgtgagcgtgaaccccggcaagaattcctgcagcccgggggatcgagagtgtcgtagaaagaagaaagaatatgtgaaatgtttagaaaacagagtggcagtgcttgaaaatcaaaacaagacattgattgaggagctaaaagcacttaaggacctttactgccacaaatcagatggtgatgctgcagcagatggttatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 19 - DNA, 2133 bp, Artificial Sequence
  Chimeric sequences
atgaaggcaatactagtagttctgctatatacatttgcaaccgcaaatgcagacacattatgtataggtggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggrtcragaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggggtgatgctgcagcagatggttatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 20 - DNA, 1714 bp, Artificial Sequence
  Chimeric sequences
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggatgaaacaacttgaagacaaggttgaagaattgctttcgaaaaattatcacttggaaaatgaggttgccagattaaagaaattagttggcgaacgcagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccatag

SEQ ID NO: 21 - DNA, 1248 bp, Artificial Sequence
  Chimeric sequences
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggatgaaacaacttgaagacaaggttgaagaattgctttcgaaaaattatcacttggaaaatgaggttgccagattaaagaaattagttggcgaacgc

SEQ ID NO: 22 - DNA, 960 bp, Artificial Sequence
  Chimeric sequences
atgaagaccatcattgctttgagctacattttctgtctggctctcggcgggagatctgacaaaactcacacatgcccaccgtgcccagcacctgaactcctggcgggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggtgatgctgcagcagatggtggattcctcccttatgttatgcccctattaggccctttgctttgcttattgcttgtgttatctttcggtccaattattttcaacaagcttatgacctttattaaacatcaaattgagagcatccaggccaaacctatacaagtccattatcatcgccttgaacaagaagacagtggtggctcatatttgaccttaacatag

SEQ ID NO: 23 - DNA, 1782 bp, Artificial Sequence
  Chimeric sequences
atggccatcatttatctcattctcctgttcacagcagtgagagggaatgacaacagcacggcaacgctgtgccttgggcaccatgcagtaccaaacggaacgatagtgaaaacaatcacgaatgaccaaattgaagttactaatgctactgagctggttcagagttcctcaacaggtgaaatatgcgacagtcctcatcagatccttgatggaaaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagcttcaattggactggagtcactcaaaacggaacaagctctgcttgcataaggagatctaaaaacagtttctttagtagattgaattggttgacccacttaaacttcaaatacccagcattgaacgtgactatgccaaacaatgaacaatttgacaaattgtacatttggggggttcaccacccgggtacggacaaagaccaaatcttcctgtatgctcaagcatcaggaagaatcacagtctctaccaaaagaagccaacaaaccgtaagcccgaatatcggatctagacccagagtaaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaattctgaatgcatcactccaaatggaagcattcccaatgacaaaccattccaaaatgtaaacaggatcacatacggggcctgtcccagatatgttaagcaaaacactctgaaattggcaacagggatgcgaaatgtaccagagaaacaaactagaggcatatttggcgcaatcgcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatagattgatcgggaaaaccaacgagaaattccatcagattgaaaaagaattctcagaagtcgaagggagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagagatgaagcattaaacaaccggtttcagatcaagggagttgagctgaagtcagggggtgatgctgcagcagatggtggattcctcccttatgttatgcccctattaggccctttgctttgcttattgcttgtgttatctttcggtccaattattttcaacaagcttatgacctttattaaacatcaaattgagagcatccaggccaaacctatacaagtccattatcatcgccttgaacaagaagacagtggtggctcatatttgaccttaacatag

SEQ ID NO: 24 - DNA, 1752 bp, Artificial Sequence
  Chimeric sequences
atggggcaagaattaagccagcatgaacgttatgtagaacaattgaagcaggctttaaagacacggggagtaaaggttaaatatgctgatcttttgaaattttttgattttgtgaaggatacttgtccttggtttccgcaagagggaaccatagatattaaacggtggcgtagagtaggcgactgtttccaagactattacaatacttttggcccggagaaagtcccagtaactgcattctcttactggaacttaattaaagaattgatagataagaaagaagttaacccacaagtaatggctgccgtagcccaaactgaagaaattttaaaaagtaattctcaaacagacctcacaaagacctctcaaaacccagacttggaccttatttcccttgatagcgacgatgaaggagctaaaagttcctctctacaagataaaggtttatcaagtactaaaaaaccaaaaagattcccagttctgttaacagcacaaactagtaaagaccctgaagaccccaacccctcagaggtagactgggacggcttggaagatgaggcagcgaaatatcataatcccgattggcctcccttcctaacccgtccacctccttacaataaagctactccttccgcacccactgtaatggcggttgttaatccaaaagaggagctcaaagaaaaaattgctcaattagaggaacagattaaattagaagagttacaccaggcactaatttccaaattacaaaaactaaaaacagggaatgaaactgtaactcacccagacacagcaggaggcctttctcgcacgcctcactggccagggcaacatatccctaaaggaaaatgttgcgccagtcgagaaaaggaagaacaaatcccaaaagatattttcccagtgactgaaaccgttgatgggcaaggtcaagcctggagacaccataatggttttgattttgccgtcataaaagaattaaaaacagctgcttcccaatatggggctactgccccatacacattagccatagtggaatctgtagcggacaattggcttacccctacagattggaatacgcttgttagggcagtcctctcaggaggagatcacttactgtggaaatctgagttttttgaaaattgcagagatacggctaaaagaaaccaacaagccggtaatggctgggattttgacatgttaacaggttcgggtaattattccagcaccgatgcacaaatgcagtatgatccaggattgtttgctcaaattcaagcggctgctacaaaagcctggagaaaacttcccgttaagggagacccaggagcctcccttacaggagtcaaacaaggacccgatgagccatttgcagatttcgtacacagacttataacaactgctgggagaatctttggaagtgctgaggccggtgtagactatgtaaaacaactagcatatgaaaatgctaatccagcttgtcaggcagccattcgcccctatagaaagaagacagatttaactggctatatccgtctttgctcggatattgggccctcttatcagcaaggcctggccatggccgccgcctttagcgggcagactgtaaaagattttcttaacaacaaaaataaagagaaaggagggggtgctgcaggtatgaaacaacttgaagacaaggttgaagaattgctttcgaaaaattatcacttggaaaatgaggttgccagattaaagaaattagttggcgaacgctag

SEQ ID NO: 25 - DNA, 2661 bp, Artificial Sequence
  Chimeric sequences
atggggcaagaattaagccagcatgaacgttatgtagaacaattgaagcaggctttaaagacacggggagtaaaggttaaatatgctgatcttttgaaattttttgattttgtgaaggatacttgtccttggtttccgcaagagggaaccatagatattaaacggtggcgtagagtaggcgactgtttccaagactattacaatacttttggcccggagaaagtcccagtaactgcattctcttactggaacttaattaaagaattgatagataagaaagaagttaacccacaagtaatggctgccgtagcccaaactgaagaaattttaaaaagtaattctcaaacagacctcacaaagacctctcaaaacccagacttggaccttatttcccttgatagcgacgatgaaggagctaaaagttcctctctacaagataaaggtttatcaagtactaaaaaaccaaaaagattcccagttctgttaacagcacaaactagtaaagaccctgaagaccccaacccctcagaggtagactgggacggcttggaagatgaggcagcgaaatatcataatcccgattggcctcccttcctaacccgtccacctccttacaataaagctactccttccgcacccactgtaatggcggttgttaatccaaaagaggagctcaaagaaaaaattgctcaattagaggaacagattaaattagaagagttacaccaggcactaatttccaaattacaaaaactaaaaacagggaatgaaactgtaactcacccagacacagcaggaggcctttctcgcacgcctcactggccagggcaacatatccctaaaggaaaatgttgcgccagtcgagaaaaggaagaacaaatcccaaaagatattttcccagtgactgaaaccgttgatgggcaaggtcaagcctggagacaccataatggttttgattttgccgtcataaaagaattaaaaacagctgcttcccaatatggggctactgccccatacacattagccatagtggaatctgtagcggacaattggcttacccctacagattggaatacgcttgttagggcagtcctctcaggaggagatcacttactgtggaaatctgagttttttgaaaattgcagagatacggctaaaagaaaccaacaagccggtaatggctgggattttgacatgttaacaggttcgggtaattattccagcaccgatgcacaaatgcagtatgatccaggattgtttgctcaaattcaagcggctgctacaaaagcctggagaaaacttcccgttaagggagacccaggagcctcccttacaggagtcaaacaaggacccgatgagccatttgcagatttcgtacacagacttataacaactgctgggagaatctttggaagtgctgaggccggtgtagactatgtaaaacaactagcatatgaaaatgctaatccagcttgtcaggcagccattcgcccctatagaaagaagacagatttaactggctatatccgtctttgctcggatattgggccctcttatcagcaaggcctggccatggccgccgcctttagcgggcagactgtaaaagattttcttaacaacaaaaataaagagaaaggagggggtgctgcaggtatgaaacaacttgaagacaaggttgaagaattgctttcgaaaaattatcacttggaaaatgaggttgccagattaaagaaattagttggcgaacgcagggtggaggggccagccccaggccccgaaacaagcttatggggcagtcagctttgttccagccaacaaaaacaacccatttcaaagcttaccagagccaccccaggaagtgcaggattggacctctgttccacctcccacacagtattaacgcctgaaatggggccccaagcgttaagcactggaatatatggaccactgcctcccaacacttttggattaatcctaggccgaagtagcattactatgaagggtctacaagtttatccaggagtaattgataatgactataccggagaaattaaaattatggcaaaagctgttaacaatattgttactgtctctcaaggcaacagaatagctcaattaatcctcctgcctctgatcgagacagacaataaggtacaacaaccttatagaggacaaggaagttttggatcctcagacatatattgggtccaacctattacttgtcaaaaaccttccttaacattatggttagatgacaaaatgttcacaggcttaatagatacgggggctgatgtcacaattatcaagctggaggactggcctcctaattggcctataacagataccttaaccaatttaagaggaataggacaaagtaacaaccctaaacaaagttctaaatatcttacttggagagataaagaaaacaattctggtctcatcaaaccgtttgttattcctaacttacctgtcaatctttggggccgagatttactttctcaaatgaaaattatgatgtgtagccccaatgacatagtaactgctcaaatgttagcccagggctacagcccaggaaaagggttaggaaaaaaggaaaatggcattctacatcctatcccaaatcaaggacaatctaacaaaaaaggttttggaaatttttaa

SEQ ID NO: 26 - DNA, 3219 bp, Artificial Sequence
  Chimeric sequences
atggggcaagaattaagccagcatgaacgttatgtagaacaattgaagcaggctttaaagacacggggagtaaaggttaaatatgctgatcttttgaaattttttgattttgtgaaggatacttgtccttggtttccgcaagagggaaccatagatattaaacggtggcgtagagtaggcgactgtttccaagactattacaatacttttggcccggagaaagtcccagtaactgcattctcttactggaacttaattaaagaattgatagataagaaagaagttaacccacaagtaatggctgccgtagcccaaactgaagaaattttaaaaagtaattctcaaacagacctcacaaagacctctcaaaacccagacttggaccttatttcccttgatagcgacgatgaaggagctaaaagttcctctctacaagataaaggtttatcaagtactaaaaaaccaaaaagattcccagttctgttaacagcacaaactagtaaagaccctgaagaccccaacccctcagaggtagactgggacggcttggaagatgaggcagcgaaatatcataatcccgattggcctcccttcctaacccgtccacctccttacaataaagctactccttccgcacccactgtaatggcggttgttaatccaaaagaggagctcaaagaaaaaattgctcaattagaggaacagattaaattagaagagttacaccaggcactaatttccaaattacaaaaactaaaaacagggaatgaaactgtaactcacccagacacagcaggaggcctttctcgcacgcctcactggccagggcaacatatccctaaaggaaaatgttgcgccagtcgagaaaaggaagaacaaatcccaaaagatattttcccagtgactgaaaccgttgatgggcaaggtcaagcctggagacaccataatggttttgattttgccgtcataaaagaattaaaaacagctgcttcccaatatggggctactgccccatacacattagccatagtggaatctgtagcggacaattggcttacccctacagattggaatacgcttgttagggcagtcctctcaggaggagatcacttactgtggaaatctgagttttttgaaaattgcagagatacggctaaaagaaaccaacaagccggtaatggctgggattttgacatgttaacaggttcgggtaattattccagcaccgatgcacaaatgcagtatgatccaggattgtttgctcaaattcaagcggctgctacaaaagcctggagaaaacttcccgttaagggagacccaggagcctcccttacaggagtcaaacaaggacccgatgagccatttgcagatttcgtacacagacttataacaactgctgggagaatctttggaagtgctgaggccggtgtagactatgtaaaacaactagcatatgaaaatgctaatccagcttgtcaggcagccattcgcccctatagaaagaagacagatttaactggctatatccgtctttgctcggatattgggccctcttatcagcaaggcctggccatggccgccgcctttagcgggcagactgtaaaagattttcttaacaacaaaaataaagagaaaggagggggtgctgcaggtatgaaacaacttgaagacaaggttgaagaattgctttcgaaaaattatcacttggaaaatgaggttgccagattaaagaaattagttggcgaacgctttcttaacaacaaaaataaagagggagggggtgctgcaaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactag

SEQ ID NO: 27 - DNA, 3129 bp, Artificial Sequence
  Chimeric sequences
atggggcaagaattaagccagcatgaacgttatgtagaacaattgaagcaggctttaaagacacggggagtaaaggttaaatatgctgatcttttgaaattttttgattttgtgaaggatacttgtccttggtttccgcaagagggaaccatagatattaaacggtggcgtagagtaggcgactgtttccaagactattacaatacttttggcccggagaaagtcccagtaactgcattctcttactggaacttaattaaagaattgatagataagaaagaagttaacccacaagtaatggctgccgtagcccaaactgaagaaattttaaaaagtaattctcaaacagacctcacaaagacctctcaaaacccagacttggaccttatttcccttgatagcgacgatgaaggagctaaaagttcctctctacaagataaaggtttatcaagtactaaaaaaccaaaaagattcccagttctgttaacagcacaaactagtaaagaccctgaagaccccaacccctcagaggtagactgggacggcttggaagatgaggcagcgaaatatcataatcccgattggcctcccttcctaacccgtccacctccttacaataaagctactccttccgcacccactgtaatggcggttgttaatccaaaagaggagctcaaagaaaaaattgctcaattagaggaacagattaaattagaagagttacaccaggcactaatttccaaattacaaaaactaaaaacagggaatgaaactgtaactcacccagacacagcaggaggcctttctcgcacgcctcactggccagggcaacatatccctaaaggaaaatgttgcgccagtcgagaaaaggaagaacaaatcccaaaagatattttcccagtgactgaaaccgttgatgggcaaggtcaagcctggagacaccataatggttttgattttgccgtcataaaagaattaaaaacagctgcttcccaatatggggctactgccccatacacattagccatagtggaatctgtagcggacaattggcttacccctacagattggaatacgcttgttagggcagtcctctcaggaggagatcacttactgtggaaatctgagttttttgaaaattgcagagatacggctaaaagaaaccaacaagccggtaatggctgggattttgacatgttaacaggttcgggtaattattccagcaccgatgcacaaatgcagtatgatccaggattgtttgctcaaattcaagcggctgctacaaaagcctggagaaaacttcccgttaagggagacccaggagcctcccttacaggagtcaaacaaggacccgatgagccatttgcagatttcgtacacagacttataacaactgctgggagaatctttggaagtgctgaggccggtgtagactatgtaaaacaactagcatatgaaaatgctaatccagcttgtcaggcagccattcgcccctatagaaagaagacagatttaactggctatatccgtctttgctcggatattgggccctcttatcagcaaggcctggccatggccgccgcctttagcgggcagactgtaaaagattttcttaacaacaaaaataaagagaaaggagggggtgctgcaggtatgaaacaacttgaagacaaggttgaagaattgctttcgaaaaattatcacttggaaaatgaggttgccagattaaagaaattagttggcgaacgctttcttaacaacaaaaataaagagggagggggtgctgcatccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccagggcttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtagagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 28 - DNA, 4020 bp, Artificial Sequence
  Chimeric sequences
atggggcaagaattaagccagcatgaacgttatgtagaacaattgaagcaggctttaaagacacggggagtaaaggttaaatatgctgatcttttgaaattttttgattttgtgaaggatacttgtccttggtttccgcaagagggaaccatagatattaaacggtggcgtagagtaggcgactgtttccaagactattacaatacttttggcccggagaaagtcccagtaactgcattctcttactggaacttaattaaagaattgatagataagaaagaagttaacccacaagtaatggctgccgtagcccaaactgaagaaattttaaaaagtaattctcaaacagacctcacaaagacctctcaaaacccagacttggaccttatttcccttgatagcgacgatgaaggagctaaaagttcctctctacaagataaaggtttatcaagtactaaaaaaccaaaaagattcccagttctgttaacagcacaaactagtaaagaccctgaagaccccaacccctcagaggtagactgggacggcttggaagatgaggcagcgaaatatcataatcccgattggcctcccttcctaacccgtccacctccttacaataaagctactccttccgcacccactgtaatggcggttgttaatccaaaagaggagctcaaagaaaaaattgctcaattagaggaacagattaaattagaagagttacaccaggcactaatttccaaattacaaaaactaaaaacagggaatgaaactgtaactcacccagacacagcaggaggcctttctcgcacgcctcactggccagggcaacatatccctaaaggaaaatgttgcgccagtcgagaaaaggaagaacaaatcccaaaagatattttcccagtgactgaaaccgttgatgggcaaggtcaagcctggagacaccataatggttttgattttgccgtcataaaagaattaaaaacagctgcttcccaatatggggctactgccccatacacattagccatagtggaatctgtagcggacaattggcttacccctacagattggaatacgcttgttagggcagtcctctcaggaggagatcacttactgtggaaatctgagttttttgaaaattgcagagatacggctaaaagaaaccaacaagccggtaatggctgggattttgacatgttaacaggttcgggtaattattccagcaccgatgcacaaatgcagtatgatccaggattgtttgctcaaattcaagcggctgctacaaaagcctggagaaaacttcccgttaagggagacccaggagcctcccttacaggagtcaaacaaggacccgatgagccatttgcagatttcgtacacagacttataacaactgctgggagaatctttggaagtgctgaggccggtgtagactatgtaaaacaactagcatatgaaaatgctaatccagcttgtcaggcagccattcgcccctatagaaagaagacagatttaactggctatatccgtctttgctcggatattgggccctcttatcagcaaggcctggccatggccgccgcctttagcgggcagactgtaaaagattttcttaacaacaaaaataaagagaaaggagggggtgctgcaggtcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattttcttaacaacaaaaataaagagggagggggtgctgcaacggagggcacgtgtctgcggcgccgagggggcccctacaagaccgagcccgccaccgacctcggccgctggcgactcaactgcgagaggggccggcagacgtggacctacctgcaggacgagcgcgccggccgcgagcagaccggcctggaagcctacgccctggggctggacaccaagaattactttaaggacttgcccaaagcccacaccgcctttgagggggctctgaacgggatgacattttacgtggggctgcaggctgaggatgggcactggacgggtgattatggtggcccacttttcctcctgccaggcctcctgatcacttgccacgtggcacgcatccctctgccagccggatacagagaagagattgtgcggtacctgcggtcagtgcagctccctgacggtggctggggcctgcacattgaggataagtccaccgtgtttgggactgcgctcaactatgtgtctctcagaattctgggtgttgggcctgacgatcctgacctggtacgagcccggaacattcttcacaagaaaggtggtgctgtggccatcccctcctgggggaagttctggctggctgtcctgaatgtttacagctgggaaggcctcaataccctgttcccagagatgtggctgtttcctgactgggcaccggcacacccctccacactctggtgccactgccggcaggtgtacctgcccatgagctactgctacgccgttcggctgagtgccgcggaagacccgctggtccagagcctccgccaggagctctatgtggaggacttcgccagcattgactggctggcgcagaggaacaacgtggcccccgacgagctgtacacgccccacagctggctgctccgcgtggtatatgcgctcctcaacctgtatgagcaccaccacagtgcccacctgcggcagcgggccgtgcagaagctgtatgaacacattgtggccgacgaccgattcaccaagagcatcagcatcggcccgatctcgaaaaccatcaacatgcttgtgcgctggtatgtggacgggcccgcctccactgccttccaggagcatgtctccagaatcccggactatctctggatgggccttgacggcatgaaaatgcagggcaccaacggctcacagatctgggacaccgcattcgccatccaggctctgcttgaggcgggcgggcaccacaggcccgagttttcgtcctgcctgcagaaggctcatgagttcctgaggctctcacaggtcccagataaccctcccgactaccagaagtactaccgccagatgcgcaagggtggcttctccttcagtacgctggactgcggctggatcgtttctgactgcacggctgaggccttgaaggctgtgctgctcctgcaggagaagtgtccccatgtcaccgagcacatccccagagaacggctctgcgatgctgtggctgtgctgctgaacatgagaaatccagatggagggttcgccacctatgagaccaagcgtggggggcacttgctggagctgctgaacccctcggaggtcttcggggacatcatgattgactacacctatgtggagtgcacctcagccgtgatgcaggcgcttaagtatttccacaagcgtttcccggagcacagggcagcggagatccgggagaccctcacgcagggcttagagttctgtcggcggcagcagagggccgatggctcctgggaaggctcctggggagtttgcttcacctacggcacctggtttggcctggaggccttcgcctgtatggggcagacctaccgagatgggactgcctgtgcagaggtctcccgggcctgtgacttcctgctgtcccggcagatggcagacggaggctggggggaggactttgagtcctgcgaggagcggcgttatttgcagagtgcccagtcccagatccataacacatgctgggccatgatggggctgatggccgttcggcatcctgacatcgaggcccaggagagaggagtccggtgtctacttgagaaacagctccccaatggcgactggccgcaggaaaacattgctggggtcttcaacaagtcctgtgccatctcctacacgagctacaggaacatcttccccatctgggccctcggccgcttctcccagctgtaccctgagagagcccttgctggccacccctga

SEQ ID NO: 29 - DNA, 2214 bp, Artificial Sequence
  Chimeric sequences
atgaagaccatcattgctttgagctacattttctgtctggctctcgctcaaaaacttcctggaagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacctccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgccggtgatgctgcagcagatggtgggatattcgggggaataggggaatgggccgttcatctgctaaaaggactgcttttggggcttgtagttattttattgctactggtgtgcctgccttgccttttacaatttgtgtctagtagtattcgaaagatgattaatagttcaatcaactatcatactgaatacaggaagatgcagggcggagcagtctag

SEQ ID NO: 30 - DNA, 1797 bp, Artificial Sequence
  Chimeric sequences
atggaaagaatagtgattgccctcgcaataatcagcattgtcaaaggtgaccaaatttgcattggttaccatgcaaacaattcaacagagcaggttgatacaatcatggaaaagaatgtgacggtcacacatgctcagaatatactggaaaaagagcacaatgggaaactttgcagtcttaaaggagtgaggcccctcattctgaaggattgcagtgtagctggatggcttcttggaaacccaatgtgtgatgaattcctgaatgtaccagaatggtcatacatcgtggaaaaagataatccagtcaatggcctgtgctatccaggagacttcagcgactacgaagaactgaagcatttaatgagcagcacaaaccattttgagaaaattcagataatccctaggagttcttggtccaatcatgatgcctcatcaggagtgagttcggcatgcccatacaatggtagatcttccttattcaggaatgtagtgtggttgatcaagaagaataatgcgtacccaacagtaaagaggacctataacaacaccaatgtagaagaccttttaataatatggggaatacaccaccctaatgatgcagctgaacaaacaaaactctaccagaactcgaacacttatgtgtctgtaggaacatcaacactgaaccagagatcaatcccagaaatagccactagacccaaagtgaacggacaaagtggaagaatggaatttttctggacaatactgaagtcgaacgatgcaatcagctttgaaagtaatgggaattttatagctcctgaatatgcgtacaaaattgccaagaaaggagattcagcaatcatgagaagtgaattggagtatggtaactgtgacaccaaatgtcagactccattgggtgctataaattccagtatgcccttccacaatgttcatcctcttaccattggggagtgccccaagtatgtcaaatcggacaaactggtccttgcaacaggactaagaaacgtaccccaaagagaaacaagaggcctatttggtgcaatagcaggattcatagaaggaggatggcaaggaatggttgacgggtggtacggataccatcatagcaatgagcagggaagtggatatgctgcagacaaagaatctacccagaaagcaatcgatgggatcaccaatagagtaaactcaatcattgacaaaatgaacactcaattcgaagccgttgggaaagaattcaacaacctagaaaggagaatagaaaatttgaataagaaaatggaagatgggtttttagatgtatggacttacaatgcagaacttctcgtgctcatggaaaacgaaagaactctggatttccatgattcaaatgtcaagaacctatacgataaggtccgactccagctgagagacaatgcaaaagaattgggcaacggatgcttcgaattctaccacaagtgtgacaatgaatgcatggaaagtgtgagaaatggaacgtatgactatccgcaatattcagaagaatcaagactgaacagagaggaaatagacggagtcaaattggaatcaatgggcacctatcagggtgatgctgcagcagatggtgggatattcgggggaataggggaatgggccgttcatctgctaaaaggactgcttttggggcttgtagttattttattgctactggtgtgcctgccttgccttttacaatttgtgtctagtagtattcgaaagatgattaatagttcaatcaactatcatactgaatacaggaagatgcagggcggagcagtctag

SEQ ID NO: 31 - DNA, 1959 bp, Artificial Sequence
  Chimeric sequences
atggaagccgtcataaaggtgatttcgtccgcgtgtaaaacctattgcgggaaaacctctccttctaagaaggaaataggggccatgttgtccctcttacaaaaggaagggttgcttatgtctccctcagacttatattccccggggtcctgggatcccattaccgcggcgctatcccagcgggctatgatacttgggaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagcaacggagcggcgaatcgacaaaggggaggaagtgggagaaacaactgtgcagcgagatgcgaagatggcgccggaggaaacggccacacctaaaaccgttggcacatcctgctatcattgcggaacagctattggctgtaattgcgccacagcctcggctcctcctcctccttatgtggggagtggtttgtatccttccctggcgggggtgggagagcagcagggccaggggggtgacacacctccgggggcggaacagtcaagggcggagccagggcatgcgggtcaggctcctgggccggccctgactgactgggcaagggtcagggaggagcttgcgagtactggtccgcccgtggtggccatgcctgtagtgattaagacagagggacccgcttggacccctctggagccaaaattgatcacaagactggctgatacggtcaggaccaagggcttacgatccccgattactatggcagaagtggaagcgcttatgtcctccccgctgctgccgcatgacgtcacgaatctaatgagagttattttagggcctgccccatatgccttatggatggacgcttggggagtccaactccagacagttatagcggcagccactcgcgacccccgacacccagcgaacggtcaagggcggggggaacggactaatttgaatcgcttaaagggcttagctgatgggatggtgggcaacccacagggtcaggccgcattattaagaccgggggaattggttgctattacggcgtcggctctccaggcgtttagagaggttgcccggctggcggaacctgcaggtccatgggcggacatcatgcagggaccatctgagtcctttgttgattttgccaatcggcttataaaggcggttgaggggtcagatctcccgccttccgcgcgggctccggtgatcattgactgctttaggcagaagtcacagccagatattcagcagcttatacggacagcaccctccacgctgaccaccccaggagagataattaaatatgtgctagacaggcagaagactgcccctcttacggatcaaggcatagccgcggccatgtcgtctgctatccagcccttaattatggcaggtgctgcaggtatgaaacaacttgaagacaaggttgaagaattgctttcgaaaaattatcacttggaaaatgaggttgccagattaaagaaattagttggcgaacgcgccgtctcgttagcgatgacaatggaacataaagatcgccccttggttagggtcattctgactaacactgggagtcatccggtcaaacagcgttcggtgtatatcaccgcgctgttggactctggagcggacatcactattatttcagaggaggattggcccaccgattggccagtgatggaggccgcgaacccgcagatccatgggataggagggggaattcccatgcgaaaatctcgtgacatgatagagttgggggttattaaccgagacgggtctttggagcgacccctgctcctcttccccgcagtagctatggttagagggagtatcctaggaagagattgtctgcagggcctagggctccgcttgacaaatttatag

SEQ ID NO: 32 - DNA, 2015 bp, Artificial Sequence
  Chimeric sequences
atggaagccgtcataaaggtgatttcgtccgcgtgtaaaacctattgcgggaaaacctctccttctaagaaggaaataggggccatgttgtccctcttacaaaaggaagggttgcttatgtctccctcagacttatattccccggggtcctgggatcccattaccgcggcgctatcccagcgggctatgatacttgggaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagcaacggagcggcgaatcgacaaaggggaggaagtgggagaaacaactgtgcagcgagatgcgaagatggcgccggaggaaacggccacacctaaaaccgttggcacatcctgctatcattgcggaacagctattggctgtaattgcgccacagcctcggctcctcctcctccttatgtggggagtggtttgtatccttccctggcgggggtgggagagcagcagggccaggggggtgacacacctccgggggcggaacagtcaagggcggagccagggcatgcgggtcaggctcctgggccggccctgactgactgggcaagggtcagggaggagcttgcgagtactggtccgcccgtggtggccatgcctgtagtgattaagacagagggacccgcttggacccctctggagccaaaattgatcacaagactggctgatacggtcaggaccaagggcttacgatccccgattactatggcagaagtggaagcgcttatgtcctccccgctgctgccgcatgacgtcacgaatctaatgagagttattttagggcctgccccatatgccttatggatggacgcttggggagtccaactccagacagttatagcggcagccactcgcgacccccgacacccagcgaacggtcaagggcggggggaacggactaatttgaatcgcttaaagggcttagctgatgggatggtgggcaacccacagggtcaggccgcattattaagaccgggggaattggttgctattacggcgtcggctctccaggcgtttagagaggttgcccggctggcggaacctgcaggtccatgggcggacatcatgcagggaccatctgagtcctttgttgattttgccaatcggcttataaaggcggttgaggggtcagatctcccgccttccgcgcgggctccggtgatcattgactgctttaggcagaagtcacagccagatattcagcagcttatacggacagcaccctccacgctgaccaccccaggagagataattaaatatgtgctagacaggcagaagactgcccctcttacggatcaaggcatagccgcggccatgtcgtctgctatccagcccttaattatggcaggtgctgcaggtcgagagtgtcgtagaaagaagaaagaatatgtgaaatgtttagaaaacagagtggcagtgcttgaaaatcaaaacaagacattgattgaggagctaaaagcacttaaggacctttactgccacaaatcagatgccgtctcgttagcgatgacaatggaacataaagatcgccccttggttagggtcattctgactaacactgggagtcatccggtcaaacagcgttcggtgtatatcaccgcgctgttggactctggagcggacatcactattatttcagaggaggattggcccaccgattggccagtgatggaggccgcgaacccgcagatccatgggataggagggggaattcccatgcgaaaatctcgtgacatgatagagttgggggttattaaccgagacgggtctttggagcgacccctgctcctcttccccgcagtagctatggttagagggagtatcctaggaagagattgtctgcagggcctagggctccgcttgacaaatttatagggagggccactgttctctag

SEQ ID NO: 33 - DNA, 3063 bp, Artificial Sequence
  Chimeric sequences
atggaagccgtcataaaggtgatttcgtccgcgtgtaaaacctattgcgggaaaacctctccttctaagaaggaaataggggccatgttgtccctcttacaaaaggaagggttgcttatgtctccctcagacttatattccccggggtcctgggatcccattaccgcggcgctatcccagcgggctatgatacttgggaaatcgggagagttaaaaacctggggattggttttgggggcattgaaggcggctcgagaggaacaggttacatctgagcaagcaaagttttggttgggattagggggagggagggtctctcccccaggtccggagtgcatcgagaaaccagcaacggagcggcgaatcgacaaaggggaggaagtgggagaaacaactgtgcagcgagatgcgaagatggcgccggaggaaacggccacacctaaaaccgttggcacatcctgctatcattgcggaacagctattggctgtaattgcgccacagcctcggctcctcctcctccttatgtggggagtggtttgtatccttccctggcgggggtgggagagcagcagggccaggggggtgacacacctccgggggcggaacagtcaagggcggagccagggcatgcgggtcaggctcctgggccggccctgactgactgggcaagggtcagggaggagcttgcgagtactggtccgcccgtggtggccatgcctgtagtgattaagacagagggacccgcttggacccctctggagccaaaattgatcacaagactggctgatacggtcaggaccaagggcttacgatccccgattactatggcagaagtggaagcgcttatgtcctccccgctgctgccgcatgacgtcacgaatctaatgagagttattttagggcctgccccatatgccttatggatggacgcttggggagtccaactccagacagttatagcggcagccactcgcgacccccgacacccagcgaacggtcaagggcggggggaacggactaatttgaatcgcttaaagggcttagctgatgggatggtgggcaacccacagggtcaggccgcattattaagaccgggggaattggttgctattacggcgtcggctctccaggcgtttagagaggttgcccggctggcggaacctgcaggtccatgggcggacatcatgcagggaccatctgagtcctttgttgattttgccaatcggcttataaaggcggttgaggggtcagatctcccgccttccgcgcgggctccggtgatcattgactgctttaggcagaagtcacagccagatattcagcagcttatacggacagcaccctccacgctgaccaccccaggagagataattaaatatgtgctagacaggcagaagactgcccctcttacggatcaaggcatagccgcggccatgtcgtctgctatccagcccttaattatggcaggtgctgcaggtcgagagtgtcgtagaaagaagaaagaatatgtgaaatgtttagaaaacagagtggcagtgcttgaaaatcaaaacaagacattgattgaggagctaaaagcacttaaggacctttactgccacaaatcagatggtgctgcaggtccagcagtatcacttgcaatgacaggtgctgcaggtgtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactga

(19) United States
(12) Patent Application Publication
(10) Pub. No.: US 2013/0315938 A1
(43) Pub. Date: Nov. 28, 2013

(54) Delivering polypeptides to phagocytes

(71) Applicant: Jeffrey N. Sfakianos, Burlingame, CA (US)

(72) Inventor: Jeffrey N. Sfakianos, Burlingame, CA (US)

(21) Appl. No.: 13/481,434

(22) Filed: May 25, 2012

(51) Int. Cl. C12N 15/11; C12N 5/10; A61P 37/04; C12N 1/19; A61K 31/7088; C12N 1/21

(52) U.S. Cl. 424/184.1; 536/23.1; 435/252.3; 435/254.2; 435/348; 435/349; 435/325

(57) ABSTRACT

The current invention is a particle comprising an assembly of lipid-enveloped core polypeptides and transmembrane polypeptides. The transmembrane polypeptides comprise sequences that bind to phagocytes through indirect or direct binding by surface receptors. The result of transmembrane polypeptide binding is engulfment by the phagocyte to low-pH compartments. The low pH triggers a conformational change in transmembrane polypeptides whereby lipids of particles and phagocytes are fused and the contents of the particle, including a polypeptide to be delivered to the cell, are released into the phagocyte.

### RELATED U.S. APPLICATION DATA

U.S. Pat. No. 6,294,165—2001 Sep. 25, Andrew Michael Lindsay Lever and Eric Hunter.
U.S. Pat. No. 5,747,307—1998 May 5, Andrew Michael Lindsay Lever and Eric Hunter.
U.S. Pat. No. 5,849,586—1998 Dec. 15, Michael Kriegler and Carl F. Perez.

### BACKGROUND

Biologicals, consisting of polypeptides or nucleic acids, are used therapeutically to alter the behavior of cells. For example, insulin increases the cellular uptake of glucose and anti-EGFR reduces the rate of cellular replication. The polypeptides are also used to treat diabetics and cancer. Further, expression of exogenous nucleic acids are being developed as gene therapies for numerous diseases. However, cells have evolved as lipid-bound compartments where the types of molecules moving across lipids, and thus between compartments, is stringently regulated. The initial regulation occurs at a limiting plasma membrane that separates the outside of the cell from the inner molecules and organelles. While the plasma membrane is semipermeable, the molecules that are allowed to cross have exquisitely co-evolved with transport molecules or satisfy a strict range of hydrophobicity profiles. Thus, biological-based therapies are currently restricted to targeting those molecules accessible from the outside of the cell. Of the examples stated above, insulin and anti-EGFR bind to the extracellular domain of transmembrane receptors and signal pathways are activated to convey the binding event to the inside of cells. Unfortunately, extracellular domains represent a minority of gene products potentially targeted with biologicals.

Accessing the inside of cells requires special packaging into vectors that allows penetration across the plasma membrane. However, vectors frequently forgo specificity of targeting particular cell types and are often immunogenic. While targeting specific cells is necessary for effective therapy, the latter problem often precludes development of solutions to the prior. When vector-based biologicals are administered to an organism, the host immune system develops antibodies to domains on the outside of the vector. Upon subsequent uses, particles or components thereof are opsonized. Opsonization involves coating with antibodies, engulfment by phagocytes, and then degradation. This process effectively decreases available vectors or doses. Efforts to overcome this problem by increasing doses promotes sepsis, leading to painful toxicity and sometimes death (N. Engl. J. Med. (2003)348:255-256 and Mol. Gen. Metab. (2003)80:148-158). Thus, the field has commonly viewed the host immune system as an obstacle to using biologicals.

For the foregoing reasons, which are not admitted to be prior art with respect to any version of the present invention by its mention in the background, there is a strong need for gaining access to the inside of cells with specificity in the cell types targeted, while avoiding the detriments imposed by a subject's immune system.

### SUMMARY

The invention described herein are nucleic acids encoding polypeptides packaged into lipid-enveloped particles and delivered specifically to phagocytes through direct and indirect mechanisms. A current need in the science is satisfied by including transmembrane polypeptides on the surface of the particles to mediate delivery to a specific cell type. Delivery of biologicals is by binding to receptors on the surface of phagocytes whereby engulfment of particles occur. Particles also comprise transmembrane polypeptides that facilitate movement of contents of the particles from engulfment compartments into the cytoplasm. The result is delivery of biological-based molecules to specific cell types.

The described particles could be used for a multitude of purposes including to promote wound healing, reduce atherosclerotic plaques, subside allergic reactions, stimulate immunity through vaccination, treat liver diseases, or to slow the effects of aging.

### DESCRIPTIONS OF THE DRAWINGS

These and other features, aspects, and advantages of the present invention will become better understood with regard to the following description, appended claims, and accompanying drawings where:

FIG. 1 shows a schematic of two categories of polypeptides of claim 1 and domains comprising the sequences. Organization of the domains represents the most preferred order, but other organizations may be used. Separate domains are represented with different patterns. (A) The first category are core polypeptides. Each particle comprises one or more of a plurality of core polypeptides. The core polypeptides of claim 1 comprise a lipid-binding domain (LBD), an interaction domain (I domain), a late domain (L domain), a protease domain (Pro), and a polypeptide to be delivered (p2bd). Sequences between domains comprise sequences that can be hydrolyzed by a chemical or an enzyme. (B) The nucleic acids encoding core polypeptides may use a ribosomal frame shift sequence to express domains on portions of translated core polypeptides. The schematic shows use of a ribosomal frame shift to express a core polypeptide whereby a portion of core polypeptides comprise a Pro domain. (C-G) The second category is transmembrane polypeptides. Transmembrane polypeptides comprise domains so that particles of claim 1 target low-pH compartments of phagocytes and fuse lipids of particles and phagocytes. Each particle may comprise one or more of a plurality of transmembrane polypeptides. Transmembrane polypeptides comprise a signal peptide domain (sig pep), a membrane spanning domain (msd), a cytoplasmic domain (cyto domain), and an extracellular domain. The extracellular domain comprises a low pH fusion domain, an antibody domain, a complement domain, a sequence recognized by the host immune system, or a combination or fragment thereof. Sequences of these domains may comprise fragments or mutations. Separate domains are outlined and can be represented with different patterns. (C) Some transmembrane polypeptides comprise sequences from similar sources. An example could be an Influenza Hemagglutinin gene. (D) Some transmembrane polypeptides may comprise sequences where signal peptides and extracellular domains from one source are combined with membrane spanning domains and cytoplasmic domains from other sources. (E) Some transmembrane polypeptides comprise sequences where each domain is from a different source. (F-G) Some transmembrane polypeptides comprise sequences in extracellular domains so that particles are targeted to phagocytes. Examples of this are not limited to extracellular domains comprising sequences similar to an antibody (F) or to a complement polypeptide (G) or a combination or fragment thereof. In the most preferred version of the current invention, transmembrane polypeptides comprise membrane spanning and cytoplasmic domains so that the sequences are complementary to core polypeptide lipid-binding domains.

FIG. 2 shows a legend of the schematics used in subsequent figures. The “transmembrane polypeptide LH” represents a transmembrane polypeptide of claim 5 that activates a fusogenic function when exposed to a low pH (LH) environment. The “low pH transmembrane polypeptide LH” represents a transmembrane polypeptide in a low pH environment. The “transmembrane polypeptide T” represents the transmembrane polypeptide from claim 5 comprising a sequence whereby the particle is targeted (T) to phagocytes. While the schematic represents separate polypeptides for fusogenic and targeting functions, these functions may be comprised in a single polypeptide. The “assembling core polypeptide” is a sequence comprising a function to assemble the core of the particle of claim 6. Maturation of particles are required for disassembly after or upon contacting the phagocyte. Domains of mature core polypeptides where domains have been separated by hydrolysis are represented by “hydrolytically matured core polypeptide” and “mature core polypeptide to be delivered.” The “host antibody” is an antibody added to the particle before administration, or most preferably, by a subject's immune system after administration. The “phagocyte surface receptor” represents a receptor on the surface of the phagocyte that binds to transmembrane polypeptides, either directly or indirectly. The “inside phagocytic cell” represents the inside a phagocyte.

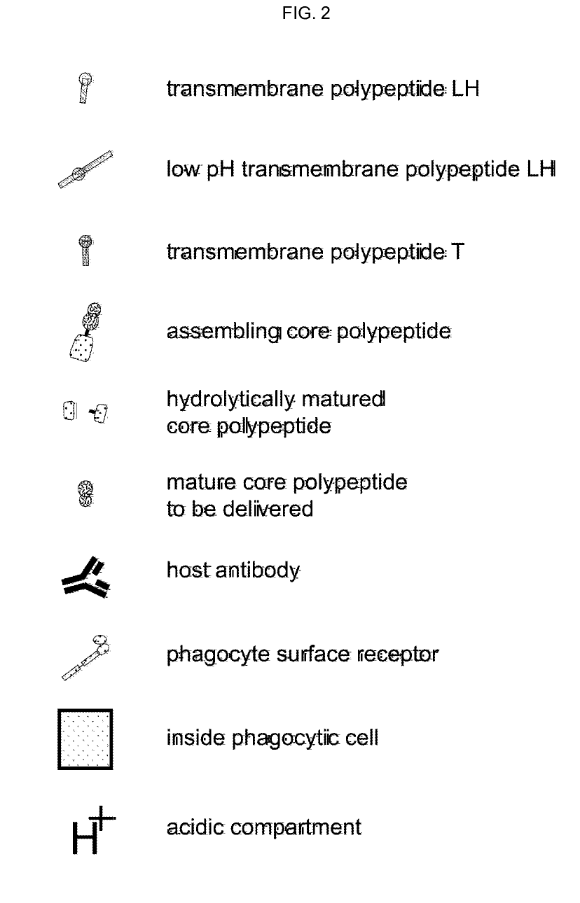

FIG. 3 shows a schematic of domains of transmembrane polypeptides and a proposed conformational change upon exposure to a low pH environment. (A) At least 2 functions of transmembrane polypeptides are claimed in the current version of the invention. One function is to target particles to phagocytes and another function is to fuse lipids of particles and phagocytes. Each function is schematically represented. Each transmembrane polypeptide comprises at least three domains, a membrane spanning domain, a cytoplasmic domain, and an extracellular domain. Membrane spanning domains are in contact with lipids and link extracellular domains to cytoplasmic domains. Cytoplasmic domains are inside of lipid-enveloped particles or contacts the cytoplasm of cells and are most preferred to complement the lipid-binding domain of core polypeptides. Signal peptides are not depicted in this schematic. Extracellular domains comprise sequences recognized by the subjects immune system, bound by antibodies, or bind directly to phagocyte surface receptors. Lipids are represented by a thickened black line extending across membrane spanning domains to represent contact between lipids and transmembrane polypeptides. (B-E) Transmembrane polypeptides T function in targeting particles to phagocytes. Panels B and C schematically represent transmembrane polypeptides T comprising sequences bound by antibodies. These sequences represent an indirect targeting to phagocytes whereby phagocyte surface receptors bind to antibodies bound to transmembrane polypeptides T of lipid-enveloped particles. Panels D and E schematically represent transmembrane polypeptides T comprising sequences binding directly to phagocyte surface receptors. A function of transmembrane polypeptides LH is to mediate mixing of lipids between particles and phagocytes. Mixing is mediated by conformational changes occurring upon exposure to a low-pH environment. The conformational changes occur whereby fusion peptides contact lipids of phagocytes. Contact disturbs equilibrium of lipid interactions whereby mixing between particle lipids and phagocyte lipids occur. This is represented in differences between the neutral pH conformation of transmembrane polypeptides LH represented in panels B and D versus the low pH conformation of transmembrane polypeptides LH represented in panels C and E. Current models of glycoprotein mediated fusion allows for hydrophobic peptide sequences to translocate from contacting lipids cis to the particle to contacting lipids trans to the particle.

FIG. 4 shows a schematic of 4 stages of delivery of polypeptides to be delivered by lipid-enveloped particles of claim 1. Lipids of producer cells, particles, and phagocytes are represented by boldened lines. The first stage (A) is an assembly of a plurality of least one of the core polypeptides of claim 4 and envelopment by a lipids comprising a plurality of least one of the transmembrane polypeptides of claim 5. This stage occurs in the cells of claim 3. Core polypeptides are matured by hydrolysis (particle maturation) so that the medium of the cell of claim 3 comprises lipid-enveloped particles of claim 1. (B) A mature lipid-enveloped particle of claim 1 is schematically represented. (C) Release of mature core polypeptides to be delivered into targeted cells involves exposure of lipid-enveloped particles to a low pH environment so that transmembrane polypeptides LH cause mixing of lipids from particles and target cells. After fusion of lipids, hydrolytically matured core polypeptides disassemble whereby mature core polypeptides to be delivered are released from particles. (D) Mature core polypeptides to be delivered are schematically represented inside a phagocyte. The preferred version of the current invention comprises mutations in polypeptides to be delivered so that the stability is altered. Increased stability is represented in (D) by a greater number of mature core polypeptides to be delivered compared to hydrolytically mature core polypeptide domains. Some applications may require destabilizing mutations.

FIG. 5 shows various states of lipid enveloped particles before fusion with phagocytes. (A) One version of the current invention uses antibodies to target a particle to phagocytes through opsonization. A lipid-enveloped particle coated with antibodies added to particles before administration or by the subject's immune system after administration is schematically represented. (B) A mature lipid-enveloped particle is represented as it exists in the medium of the cell of claim 3. This embodiment of the particle does not need to bind antibodies to target phagocytes. Transmembrane polypeptides T of this embodiment comprises sequences binding to phagocyte surface receptors. (C) A lipid-enveloped particle coated with antibodies is schematically represented binding to a phagocyte through contacts with phagocyte surface receptors. The schematic represents a version of the current invention whereby transmembrane polypeptides T comprise sequences so that phagocyte surface receptors bind particles indirectly. (D) Another version of the current invention is a particle comprising transmembrane polypeptides T comprising sequences similar to antibodies or Complement or a combination or fragment thereof so that phagocyte surface receptors bind directly to lipid-enveloped particles. Phagocyte surface receptors are schematically represented to bind directly to transmembrane polypeptides of particles of claim 1; and

FIG. 6 shows a schematic representation of particles binding directly and indirectly to a phagocyte and engulfment and exposure to a low pH environment whereby polypeptides to be delivered are released to phagocytes. (A) A lipid-enveloped particle is represented in 2 embodiments schematically separated by a line showing antibodies bound to transmembrane polypeptide T comprising sequences to which the host has been immunized on one side (marked “indirect”) and transmembrane polypeptides T comprising antibody or complement domains on the other side (marked “direct”). Both direct and indirect binding are represented prior to contacting phagocytes. This representation occurs in all panels of this figure. (B) Binding to phagocyte surface receptors is schematically represented whereby engulfment of lipid-enveloped particles begins. (C) A lipid-enveloped particle is engulfed by a phagocyte and is represented to be enclosed in a compartment. Phagocytes acidify the compartment thereby activating the fusogenic function of transmembrane polypeptide LH. (D) An activated fusogenic function of transmembrane polypeptide LH causes mixing of particle lipids with phagocyte lipids. A result is release of mature core polypeptides to be delivered into the phagocyte. While a means of transmembrane polypeptides T binding to phagocytes can be different, the result is similar. Particles are moved to compartments of increasing acidity. Lowered pH activates enzymes that degrade the contents of the compartment. However, the invention allows escape of the polypeptides before degradation as transmembrane polypeptides LH mediate fusion of lipids between particles and phagocytes so that mature core polypeptides to be delivered are released.

### DESCRIPTION

### Definitions

The term lipids can include single molecules of lipids or groups of lipids organized into a monolayer or bilayer. The lipids are comprised by any embodiment of the particles of the present invention and by an organelle of producer and target cells. The lipids of the producer and target cells are not limited to the plasma membrane, Endoplasmic Reticulum, intermediate compartment, Golgi apparatus, trans-Golgi network, endosomes, lysosomes, phagosomes, and nuclear envelope.

The term polypeptide is used to mean a sequence of more than 2 amino acids. The term may include peptides that are generally considered to be sequences of less than about 100 amino acids and proteins that are generally considered to be sequences of greater than about 100 amino acids. These amino acid sequences are linked together to form polypeptides. Polypeptides are intended to include post-translational modifications, mutations, or sequences possessing >50% homology, or preferably >60% homology, or more preferably >70% homology, or most preferably >80% homology at the amino acid level to those defined by the nucleic acid sequences in this document.

Similar to the degeneracy of nucleic acid codons in specifying an amino acid, an amino acid sequence possesses degeneracy in specifying a structural conformation of polypeptides. It is the structure of the domain that specifies the function of the domain. Thus, domains are described by functions in the claims of this document, as many sequences of amino acids may achieve the claims of the assembled functional domains. While many naturally occurring sequences are included with this document, similar results could be achieved using polypeptide sequences designed de novo that specify structural requirements, and hence functional requirements, of the invention. Thus, the sequences comprised within this document are not a limitation of the invention, but merely a guide for assembly of functional domains to deliver polypeptides to phagocytes.

Polypeptide domains include sequences of amino acids comprising at least one function. A polypeptide may comprise more than one polypeptide domain. Polypeptide domains may have different origins linked together to make a single polypeptide. A single polypeptide domain may also make a polypeptide.

The term “fragment” is used to refer to a functional or structural domain of a polypeptide or protein. An example of a fragment is not limited to an Fc domain that is comprised by an antibody.

The term “polypeptide to be delivered” is used to refer to polypeptides encoded by the nucleic acids of claim 1 as a domain comprised within a core polypeptide whereby hydrolysis releases this domain from the core polypeptide so that it functions inside phagocytes. The domain may comprise transcription factors, antibodies, scaffolds for organelle biogenesis and polypeptide complexes, kinases, lipases, or other enzymes and mutants and fragments thereof. The domain may comprise nuclear factors and chromosome binding proteins. Polypeptides to be delivered may comprise chimeric polypeptide sequences whereby more than one polypeptide domain is linked to another. Polypeptides to be delivered may also comprise a vaccine antigen whereby the sequence is presented by phagocytes to lymphocytes and other cells so that the immune system can recognize the polypeptide comprising the sequences. The polypeptide to be delivered may also comprise a chimeric or de novo designed sequence.

The term “hydrolytic” is used to refer to hydrolysis of peptide bonds broken by chemical or enzymatic means. The term is used in this document to refer to the breaking of peptide bonds between amino acids of core polypeptides whereby domains of core polypeptides are released to allow disassembly of particles and release of polypeptides to be delivered upon contacting the phagocyte.

The term fusion peptide refers to a peptide sequence comprising hydrophobic amino acids whereby the peptide has a high affinity for contacting lipids. The fusion peptide contacts lipids and causes disruption of equilibrium between lipids. The fusion peptide may be comprised within a polypeptide sequence. Hydrophobic amino acids are leucine, isoleucine, and valine.

The terms fusogenic and fusion refers to mixing of lipids. In the context of this document, the term refers to transmembrane polypeptides LH comprising a fusion peptide in the extracellular domain and a membrane spanning domain. Exposure to an acidic environment causes conformational changes in extracellular domains of transmembrane polypeptides LH whereby fusion peptides insert into lipids of phagocytes. The membrane spanning domain of transmembrane polypeptides LH contacts lipids of the particle. The linkage of the lipids of the particle to the disrupted lipid equilibrium on the phagocyte causes mixing of the lipids.

A production cell comprises the nucleic acids of claim 1 operably linked to expression control sequences so that core polypeptides and transmembrane polypeptides are expressed. Production cells can derive from bacteria, yeast, insect, fish, avian, or mammalian origins. Production cells generally culture in a medium supporting growth of the cells while lipid-enveloped particles are produced and collected from the medium.

The target cell is a cell in a mixed population for which particles have higher affinity compared to other cells. Target cells are those having high capacity for receptor-mediated phagocytosis. Target cells are most preferred to be phagocytes in a human or other organism or subject. Target cells may also be cultured in vitro.

Phagocytes are defined as cells having a high propensity to engulf lipid-enveloped particles. These cells are not limited to dendritic cells, macrophages, neutrophils, and granulocytes and the subtypes of these cells.

The term “immune recognition” is defined as a specific reaction by the host immune system to bind to molecules. In the context of this document, the molecules are any polypeptides included on particles, either directly or indirectly. Immune recognition is mediated by antibodies or a major-histocompatibility complex.

Opsonization is the alteration of surfaces of particles making the particles more easily engulfed by phagocytes. Alterations comprise binding of particles by antibodies and complement proteins. Coating particles with antibodies prior to administering particles and transmembrane polypeptides comprising antibody domains or complement domains may functionally mimic opsonization so that particles are engulfed by phagocytes.

Surface receptors are polypeptides in contact with phagocytes. Surface receptors may be encoded by the phagocyte. The receptors are in contact with a phagocyte through indirect and direct interactions and may comprise one or more membrane spanning domains.

A subject is any organism, patient, or host possessing an immune system whereby adaptive and innate immunity cooperate to remove antigens.

### Overview

The isolated nucleic acids claimed comprise sequences so that when operably linked to an expression regulatory element, such as commonly used CMV or SV40 promoters or constitutively active yeast or bacterial promoters, and transfected into cells, encoded polypeptides are expressed. Cells that express the nucleic acids become known as “producer cells.” Producer cells can be any type of cell, but some cell types are preferred. Among the more preferred cell types for producer cells are 293T, Jurkat, Chinese Hamster Ovary, and yeast cells. These cells are known to someone of ordinary skill in the science. The producer cells express the nucleic acids transiently after transfection. More preferably, stably transfected cells are selected and propagated so that the progeny cells continue to express the nucleic acids.

Encoded polypeptides comprise domains so that polypeptides assemble into particles that are enveloped by lipids of the producer cells. At least two categories of polypeptides, core polypeptides and transmembrane polypeptides, cooperate to form particles.

One polypeptide category comprises core polypeptides. Core polypeptides may comprise at least one lipid-binding domain so that polypeptides contact lipids of producer cells. Lipid-binding domains may include several features, such as covalently linked lipids and structural folds comprising a surface of basic amino acids that contact lipids.

At least one of the encoded core polypeptides may comprise at least one interaction domain so that a plurality of core polypeptides bind to each other. A plurality of interaction domains binding in trans to interaction domains comprised within other core polypeptides causes oligomerization of core polypeptides whereby an assembly of 500 to 10,000 core polypeptides is formed.

Lipid-binding and interaction domains allow core polypeptides to assemble into particles that are enveloped by lipids. Lipid-enveloped particles may be extruded from producer cells by the described oligomerization and lipid-binding events depending on the producer cell type, or may include at least one late domain so that extrusion occurs. A late domain may comprise at least one polypeptide sequence whereby a ubiquitin molecule is attached to facilitate release from producer cells.

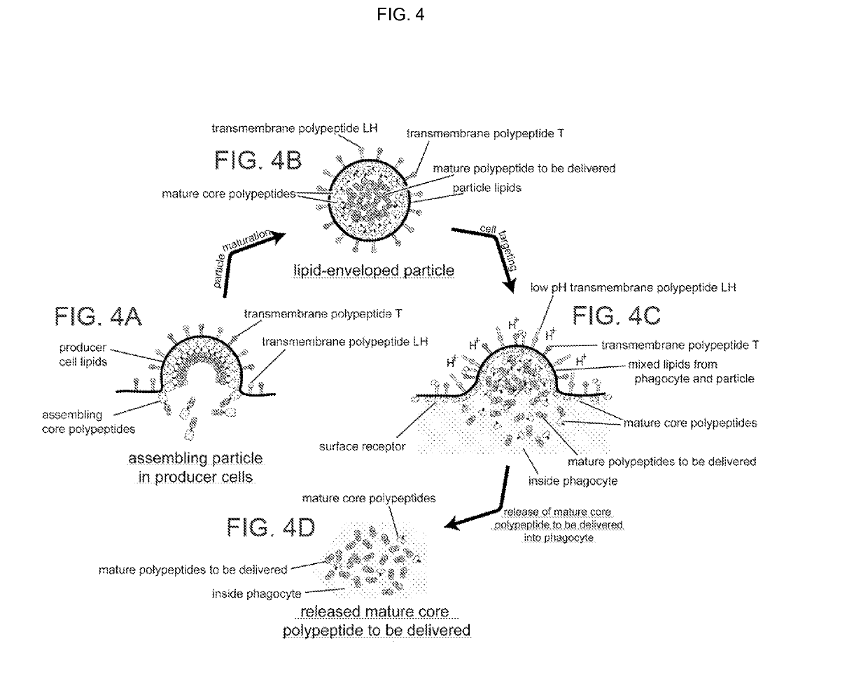

Domains function to assemble core polypeptides into lipid-enveloped particles that are released from producer cells. Other domains may be included in core polypeptides so that core polypeptides disassemble after lipids of particles and phagocytes fuse. Core polypeptides may comprise protease domains that hydrolyzes peptide bonds between specific sequences of amino acids. A plurality of these sequences hydrolyzed by a protease or chemical application may be encoded within core polypeptides whereby core polypeptides are hydrolyzed. A hydrolytic process whereby the core polypeptide is prepared for disassembly is called particle maturation (FIG. 4A and FIG. 4B).

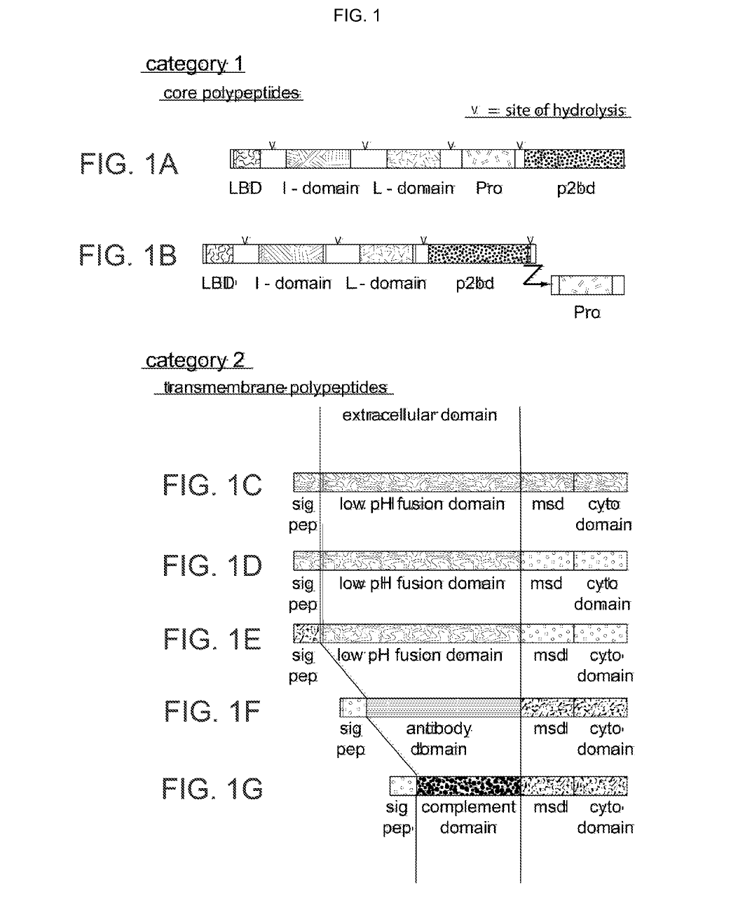

Particle maturation occurs so that core polypeptide domains can disassemble and be released to function in target cells. Core polypeptides comprise another domain whereby this domain functions at target cells. This domain comprises a polypeptide to be delivered as also described in FIG. 1. Polypeptides to be delivered comprise, but are not limited to, transcription factors, antibodies, scaffolds, antigens to function as vaccines, and chimeric and designed and engineered polypeptide sequences. Polypeptides to be delivered may also comprise fragments and mutations and post-translational modifications and mimics of post-translational modifications of the polypeptide sequences mentioned above.

Particles exclude nucleic acids unless required for the application. The exclusion of nucleic acids or the incorporation of specific nucleic acids may be quantified using polymerase chain reaction with degenerate or specific oligonucleotides. Specific nucleic acid sequences may be incorporated into particles by a polypeptide to be delivered that includes specific binding affinity for the desired sequences.

It is preferred that polypeptides to be delivered comprise mutations altering stability and half-life and activity of the polypeptides in target cells relative to naturally occurring domains with similar sequences. For example, if a transcription factor is phosphorylated at a particular serine residue by a kinase to promote ubiquitination and proteosomal degradation, then this serine residue could be mutated whereby phosphorylation does not occur and the function of the transcription factor is prolonged. Another example is a mutation of a kinase whereby the kinase activity is constitutively activated.

Another polypeptide category are transmembrane polypeptides. Transmembrane polypeptides comprise at least one signal peptide domain so that nascent polypeptide chains are cotranslationally translocated to the endoplasmic reticulum of producer cells. Signal peptide domains are linked to extracellular domains whereby extracellular domains contact the extracellular medium.

Transmembrane polypeptides may also comprise at least one cytoplasmic domain so that extracellular domains are anchored to lipid envelopes. Cytoplasmic domains are located on the cytoplasmic side of lipid envelopes. Transmembrane polypeptides may also comprise at least one membrane spanning domain so that cytoplasmic domains and extracellular domains of a transmembrane polypeptide are linked.

Versions of this invention use lipid-enveloped particles comprising a plurality of 2 transmembrane polypeptides to target to and fuse to phagocytes. The transmembrane polypeptides are designated transmembrane polypeptide T (for Targeting) and transmembrane polypeptide LH (for low pH). Different categories of transmembrane polypeptides may comprise the same sequences.

Transmembrane polypeptide LH comprises an extracellular domain whereby structural conformations of the polypeptide changes upon exposure to a low pH environment. The conformational change of transmembrane polypeptide LH allows lipids of particles to fuse with lipids of target cells. Fusion of particle and phagocyte lipids occurs so that mature core polypeptide domains are delivered to target cells.

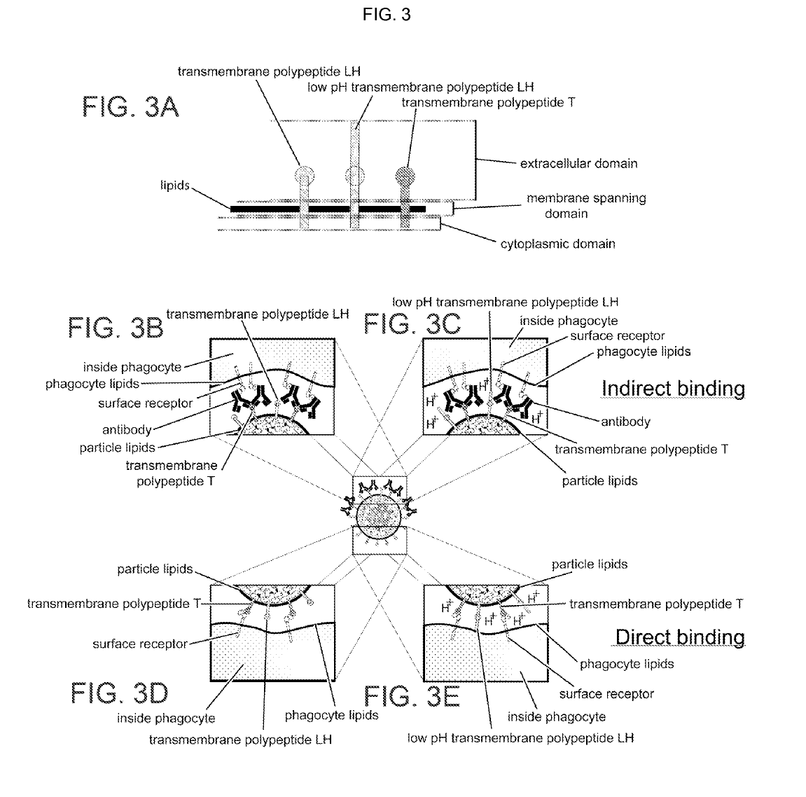

Another function of transmembrane polypeptides of the particle is targeting particles to cells whereby the cell type particles contact is specified. As described in FIG. 3, transmembrane polypeptide T may function directly or indirectly.

In one version of the current invention nucleic acids encode an extracellular domain of transmembrane polypeptides T comprising sequences similar to complement 3 or a fragment thereof, such as complement 3b or 3a whereby binding of polypeptides comprising the sequence of complement 3 or a fragment thereof to cell surface receptors causing engulfment of particles.

In another version of the current invention nucleic acids encode an extracellular domain of transmembrane polypeptides T comprising sequences similar to polypeptide sequences of Influenza Hemagglutinin to which the subject has been vaccinated. This hemagglutinin sequence may have been used in a current vaccine formulation or a vaccine formulation from a prior year. The sequence may also comprise part of a vaccination regimen. Upon administering the particle to a subject, hemagglutinin sequences are opsonized.

Examples of hemagglutinin sequences include, but are not limited to H1 as in SEQ ID NO: 001, H2 as in SEQ ID NO: 002, H3 as in SEQ ID NO: 003, H4 as in SEQ ID NO: 004, H5 as in SEQ ID NO: 005, H6 as in SEQ ID NO: 006, H7 as in SEQ ID NO: 007, H8 as in SEQ ID NO: 008, H9 as in SEQ ID NO: 009, H10 as in SEQ ID NO: 010, H11 as in SEQ ID NO: 011, H12 as in SEQ ID NO: 012, H13 as in SEQ ID NO: 013, H14 as in SEQ ID NO: 014, H15 as in SEQ ID NO: 015, and H16 as in SEQ ID NO: 016 from mammalian and avian sources. Examples of sequences are not limited to members of the Influenza A and Influenza B families as in SEQ ID NO: 017.

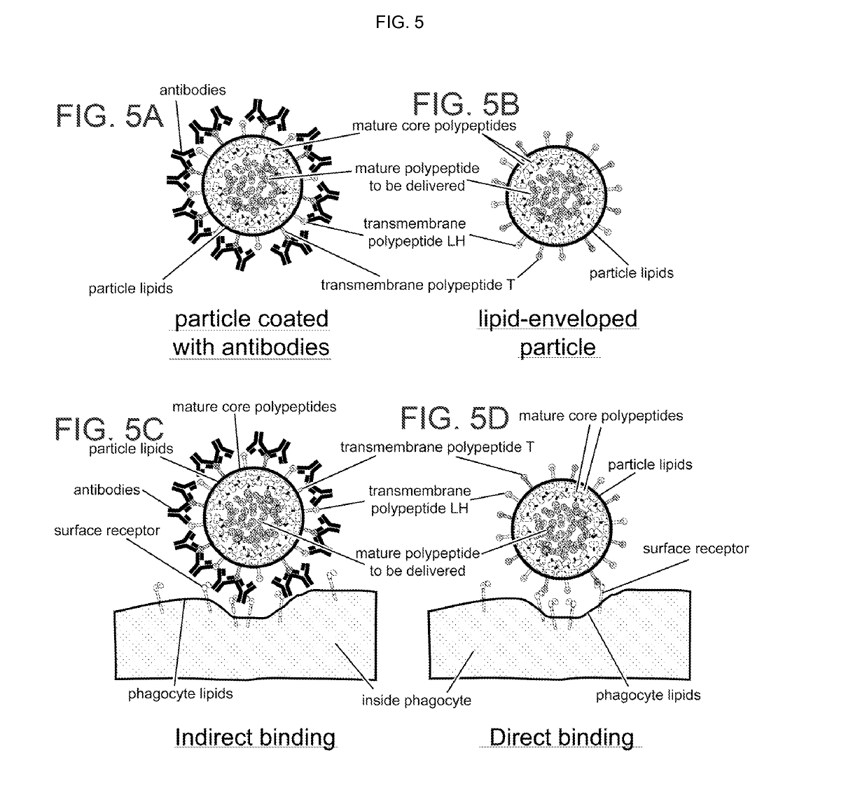

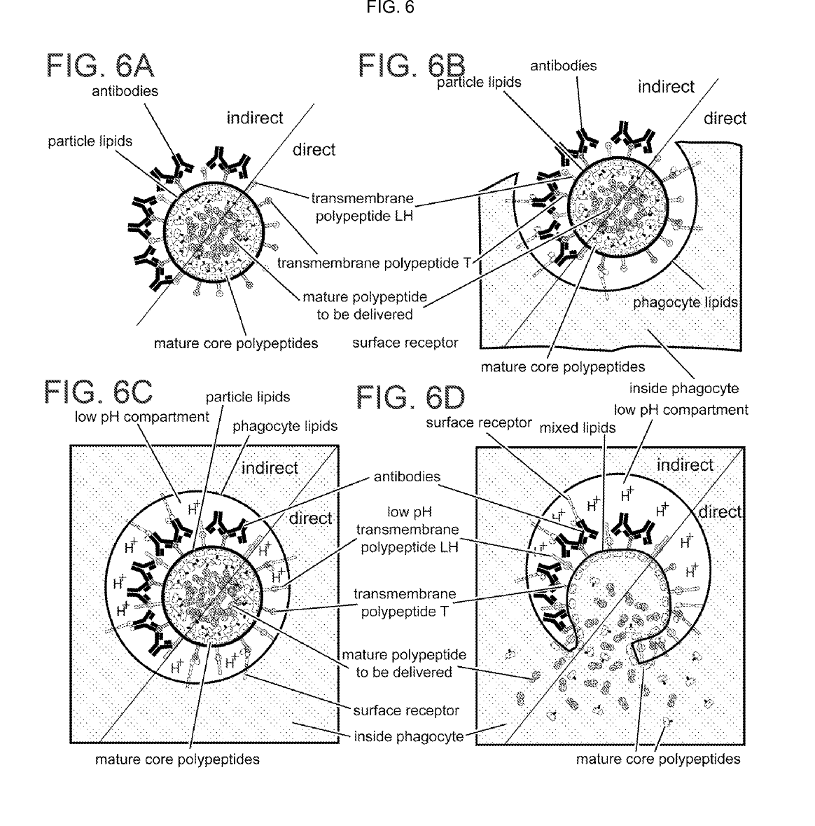

Each hemagglutinin molecule forms an oligomeric structure of 3 molecules. Each of these molecules has 2 binding sites for sialic acid. For Influenza, sialic acid acts as the cell surface receptor and because sialic acid is a common conjugate attached to molecules, the surface receptor for Influenza is on many cell types. The nucleic acids encoding transmembrane polypeptides comprising hemagglutinin sequences would most preferably comprise mutations so that binding to sialic acid is reduced. Reduction in sialic acid binding is such that the claimed indirect or direct means of targeting to phagocytes as described in FIG. 3, FIG. 5, and FIG. 6 are the dominant mode of targeting.

Membrane spanning and cytoplasmic domains of transmembrane polypeptides of the particle may comprise sequences encoding membrane spanning and cytoplasmic domains from the Influenza Hemagglutinin. Membrane spanning and cytoplasmic domains of transmembrane polypeptides may comprise sequences encoding domains that are complementary to lipid-binding domain of core polypeptides. Membrane spanning and cytoplasmic domains of transmembrane polypeptides comprise sequences encoding other transmembrane proteins as one skilled in the science would readily be able to define.

In each of these versions of the inventions described, the transmembrane polypeptide T comprises sequences whereby the particle is targeted to phagocytes by an indirect or direct binding to phagocyte surface receptors and engulfed by a phagocyte. Acidification of engulfed compartments occur whereby transmembrane polypeptides LH are conformationally changed whereby fusion of lipids release mature polypeptides to be delivered to the phagocyte.

A lipid-enveloped particle is assembled when a plurality of at least one transmembrane polypeptide is combined with a plurality of at least one core polypeptide to form an oligomeric assembly whereby the assembly is enveloped with lipids from producer cells and extruded into medium. Particles may comprise about 500 to 10,000 core polypeptides and about 100 to 5,000 transmembrane polypeptides. Some particles may comprise greater than about 10,000 core polypeptides and greater than about 5,000 transmembrane polypeptides.

### Detailed Description of the Elements

The structure of the invention allows for more than one embodiment of particles to be produced. The following description is just one example of elements operable comprised by the invention.

To begin using the inventions described herein, particles must be assembled in producer cells by expressing the nucleic acids of claim 1. Particles are assembled by expressing nucleic acids in producer cells as in claim 3. Producer cells are cultured as one of average skill in the science would easily understand. Producer cells are most commonly 293T cells when culturing producer cells in volumes less than about 5 liters. It is preferred that non-adherent producer cells be used in volumes greater than about 3 liters. Examples of non-adherent producer cells are not limited to CHO, NS/0, BHK, and 293. New cells are being developed that have desirable qualities for manufacturing volumes greater than about 3 liters and are available commercially and through license. These cells may also be transfected with the nucleic acids of claim 2 and stable transfectants generated to express the nucleic acids of claim 1 as one skilled in the science would understand. Most preferred characteristics for producer cell lines are rapidly dividing cells that can grow to at least about 20,000,000 cells per milliliter, have a capacity to produce at least about 8 picograms of secreted particles per cell per day production, are capable of continued passage under common eukaryotic cell culture conditions, and whereby the particles of claim 1 comprise greater than about 80% of the polypeptides secreted into the medium. Cells having all of these characteristics are most preferred. If production of particles at lower efficiencies can be tolerated for an application, then having all of these characteristics is not required.

The nucleic acid sequences encoding the polypeptides of claim 1 may vary depending on the need of the user and includes degenerate variants of the sequences included in this invention. The sequences can originate from any source so that when they are combined lipid-enveloped particles are assembled that deliver polypeptides to be delivered to phagocytes.

The codon usage of the nucleic acids encoding the polypeptides should be optimized for the species of the producer cell type. For example, if 293T cells are chosen as producer cells, then the nucleic acids of claim 1 should encode polypeptides using optimized codons for humans. Further, if CHO cells are chosen as producer cells, then the nucleic acids of claim 1 should encode polypeptides using optimized codons for hamsters.

The nucleic acids of claim 1 are placed into vector and operably linked to an expression control sequence, such as a CMV or SV40 promoter, and transfected into the producer cells through lipid-transfection, chemical precipitation, electroporation, or other commercially available means. The producer cells may comprise the nucleic acids transiently or, more preferably, stable transfectants selected and cloned as someone skilled in the science would easily understand. The molar ratio of nucleic acids encoding core polypeptides to nucleic acids encoding transmembrane polypeptides is typically at least about 1:1 and no more than 20:1 and at least about 1:20. If more than one sequence of transmembrane polypeptide or core polypeptide is used then the ratio should include the sum of each sequence in each category. For example, an application that requires 2 transmembrane polypeptides and 1 core polypeptide might use about 0.25 parts of each transmembrane polypeptide and about 0.50 parts of the core polypeptide.

The producer cells of claim 3 express core polypeptides and transmembrane polypeptides of claim 4 and claim 5 so that the polypeptides assemble into particles enveloped by lipids of producer cells. Particles are purified from the medium to a degree as needed for the application. For some applications the medium comprising particles is sufficient without further isolation. Purification and concentration of particles are achieved by centrifugation and chromatography as one skilled in the science would understand.

Particles can be quantified by counting particles per volume, spectrophotometric analysis, lipid mass measurement, or by SDS-PAGE analysis of polypeptides all of which are easily understood by someone skilled in the science.

### Application and Formulation

Particles can be administered to a subject as a therapy in several ways. Particles may be applied through rectal or vaginal suppository, topically, directly to a wound or abrasion, orally, subcutaneously, intravascularly, intramuscularly, and inhaled. A formulation may be one allowed by current technology and maintains the pH of particles at no less than about 7. Particles can be incorporated into a salve, lotion, paste, cream, suspension, solution, salt, powder, gel, inhalant, suppository, or capsule administered to a subject topically, inhaled, injected, by drops, or swallowed as needed for the application or therapy.

Before administration, it is critical that pH be maintained above that required to induce conformational changes to cause fusion. This pH may vary according to the transmembrane polypeptide sequence used, but conformational changes are usually induced at about pH5.5.

The current invention provides for several embodiments for targeting through transmembrane polypeptide T and fusion through transmembrane polypeptide LH. A therapy regimen may include different embodiments or particles comprising different transmembrane polypeptides T and transmembrane polypeptides LH as needed to maintain efficacy of delivery.

Samples of blood may be collected from the subject during therapy so that the degree of efficacy of delivery can be determined. This is performed by treating a small amount of the particles (an amount sufficient to deliver polypeptides to about 1,000,000 cells) with dilutions of the subject's blood. The precise amounts of particles and blood used are not important as long as a relative measurement is made with particles capable of delivering polypeptides to a target cells but not exposed to the subject's blood.

It is expected that the subject's blood will increase the amount of polypeptide delivered to phagocytes when the efficacy of delivery is through immune recognition of transmembrane polypeptides by the host.

Treating the particles with the subject's blood might also result in neutralization of the transmembrane polypeptide LH. If a reduction in efficacy of delivery results from the presence of the subject's blood then the embodiment of transmembrane polypeptide LH administered to the host should be substituted with another embodiment of transmembrane polypeptide LH having greater efficacy of delivery than the administered embodiment.

Efficacy of delivery is measured by determining percent change of polypeptides to be delivered entering phagocytes from that which is administered to cells. For example, 10,000 picograms of particles are administered to 1,000,000 phagocytes whereby 500,000 phagocytes exhibit the desired change. If the subjects blood comprises antibodies to the particle, then 70% of phagocytes might exhibit the desired change. Measurements are made using microscopy or biochemistry as allowed by the polypeptide to be delivered. The subject's blood may be tested with various embodiments of the transmembrane polypeptides to determine the optimal transmembrane polypeptides.

### EXAMPLES

### Example 1

This prospective example demonstrates using particles for a vaccine against HIV, Bacillus anthracis, and Poxviridae.

Producer cells of claim 3 are transfected to express about 0.5 to 0.0035 picomoles per 1,000,000 cells of each of two classes of transmembrane polypeptides and two classes of core polypeptides. Transmembrane polypeptides T comprise sequences similar to immunoglobin epsilon constant region similar to SEQ ID NO: 018. This version of the transmembrane polypeptide T comprises a targeting function that binds to Fc receptors on the surface of phagocytes inducing engulfment. Transmembrane polypeptides LH comprise sequences similar to H1 Hemagglutinin from H1 N1 Influenza A/California/7/2009 with mutations reducing affinity for sialic acid similar to SEQ ID NO: 019. This version of the transmembrane polypeptide LH comprises a target function through indirect binding of antibodies produced in subjects receiving the 2010 and 2011 Influenza vaccines and a fusogenic function through a low pH conformational change intrinsic to Influenza Hemagglutinins. One of the core polypeptides comprises lipid-binding, interaction, and late domains with a protease domain similar to SEQ ID NO: 020. Another core polypeptide comprises one sequence also comprising lipid-binding, interaction, and late domains with a protein to be delivered comprising a sequence from HIV-1, an anthrax epitope, and a smallpox epitope fused to a proteosomal targeting sequence similar to SEQ ID NO: 021.

Medium from cells expressing the nucleic acids comprise assembled particles. The particles are isolated by centrifugation or chromatography as one skilled in the science would understand.

Particles are treated with a pharmaceutically effective amount of lipopolysaccharide, or similar compound, whereby the molecule activates toll-like receptors in phagocytes upon contacting particles. Activation of toll-like receptors in the phagocytes promote presentation of antigens to the immune system.

Isolated particles can be quantified by counting particles per volume, spectrophotometric analysis, lipid mass measurement, SDS-PAGE analysis of polypeptides, or by functional analysis, all of which are easily understood by someone skilled in the science.

A pharmaceutically effective amount of particles are introduced into the host by rectal or vaginal suppository or inhaled using a pharmaceutically acceptable carrier or diluent formulation. Resident phagocytes along the epithelium recognize the complement 3b sequences of the particle's transmembrane polypeptide T and phagocytose the particles. Upon acidification of the phagosome, transmembrane polypeptides LH undergo conformational changes whereby the lipids of the particle and the phagosome fuse to release the contents of the particle into the phagocyte. A mass of about 100 particles should comprise a number of mature polypeptides to be delivered sufficient for a phagocyte to present the antigenic epitopes, but may be as low one particle.

A vaccination is further implemented by polypeptides to be delivered comprising an HIV sequence along with a proteosome targeting sequence so that the polypeptides to be delivered are hydrolyzed for antigen presentation by the proteosome for binding to the major histocompatibility complexes (MHC). Antigen presentation is also implemented by including lipopolysaccharide or similar compound in the particle lipids whereby the signaling pathway initiated by toll-like receptors are activated.

Transmembrane polypeptides may also comprise an HIV sequence in the extracellular domain whereby after repeated applications, antibodies can be developed by the host to the polypeptide sequence. This utilization of the invention would allow for vaccinations to be incorporated. Immunization would occur on an optimal schedule.

### Example 2

This prospective example demonstrates using particles to deliver peroxisome proliferator-activated receptor (PPAR) and liver X nuclear receptor (LXR) transcription factors whereby phagocytes change from having M1 characteristics to having M2 characteristics (Journal of Clinical Investigation, (2007) 117:89-93 and references therein).

Producer cells of claim 3 are transfected to express about 0.5 to 0.0035 picomoles per 1,000,000 cells or stably transfected and selected to express each of two classes of transmembrane polypeptides and two classes of core polypeptides. Transmembrane polypeptides T comprise a sequences similar to Fc fragments of immunoglobins similar to SEQ ID NO: 022. Transmembrane polypeptides LH comprise sequences similar to H3 Hemagglutinin from H3N2 Influenza A/Perth/16/2009 (H3N2) with mutations reducing affinity for sialic acid similar to SEQ ID NO: 023. This embodiment of the transmembrane polypeptide LH comprises targeting functions through indirect binding of antibodies produced in subjects receiving the 2011/2012 Influenza vaccines and a fusogenic functions through low pH conformational changes intrinsic to Influenza Hemagglutinins. Core polypeptides comprise lipid-binding, interaction, and late domains similar to SEQ ID NO: 024. One of the core polypeptides further comprises a protease domain similar to SEQ ID NO: 025. Another of the core polypeptides further comprises a protein to be delivered comprising sequences similar to PPAR or LXR similar to SEQ ID NO: 026 and SEQ ID NO: 027.

Medium from cells expressing the nucleic acids comprise particles. The particles are isolated by centrifugation and chromatography as one skilled in the science would understand.

Isolated particles can be quantified by counting particles per unit volume, spectrophotometric analysis, lipid mass measurement, SDS-PAGE analysis of polypeptides, or by functional qualification, all of which are easily understood by someone skilled in the science. It is expected that about 100 particles will be required to change a phagocyte from characteristics of M1 to characteristics of M2 and may be as low as 1 particle. Doses given to an organism are determined by the number of phagocytes desired to be changed and pharmaceutically optimized for the application.

Particles may be administered using a transdermal device, subcutaneously, and intravascularly using a pharmaceutically acceptable carrier and diluent formulation to a subject where phagocytes bind directly to the Fc polypeptide sequence on the transmembrane polypeptide T of the particle and indirectly through antibodies bound to the transmembrane polypeptide LH. Subjects administered particles would have all of the benefits afforded by a phagocyte having M2 characteristics. Examples of these benefits are not limited to reduction of an atherosclerotic plaque and addition of extracellular matrix to assist in tissue regeneration. Particles may also be applied topically to a wound or abrasion to promote healing.

In another embodiment of this invention core polypeptides include sequences comprising lipid-binding, interaction, and late domains with a protein to be delivered comprising a sequence similar to oxidosqualene cyclase similar to SEQ ID NO: 028. These particles could also be used to treat subjects with atherosclerosis or metabolic diseases.

### Example 3

This prospective example demonstrates using particles to deliver PPAR transcription factors to patients with liver disease and cirrhosis whereby hepatic phagocytes catabolize lipids.

Producer cells of claim 3 transfected to express about 0.5 to 0.0035 picomoles per 1,000,000 cells of each of two classes of transmembrane polypeptides and two classes of core polypeptides. The transmembrane polypeptide T comprises a sequence encoding a sequence similar to a complement 3b domain similar to SEQ ID NO: 029. Transmembrane polypeptides LH comprise sequences encoding the H5 Hemagglutinin from H5N1 Influenza A with mutations reducing affinity for sialic acid similar to SEQ ID NO: 030. This version of the transmembrane polypeptide LH comprises functions to vaccinate the patient to H5 Influenza Hemagglutinin and a fusogenic function through the low pH conformational changes of the polypeptides. In this embodiment of the current invention, the first doses to a subject are expected to be less efficacious than subsequent doses as the subject develops antibodies to the polypeptides. In another embodiment, antibodies from another source could be used to coat the particles before administration and the dosing regimens can be changed to a regimen optimally designed for the application. Core polypeptides comprise lipid-binding, interaction, and late domains similar to SEQ ID NO: 031. One of the core polypeptides further comprises a protease domain similar to SEQ ID NO: 032. Another of the core polypeptides further comprises a protein to be delivered comprising a sequence similar to PPAR as in SEQ ID NO: 033.

Medium from the cells expressing the nucleic acids of claim 1 comprise particles. Particles are isolated by centrifugation and chromatography as one skilled in the science would understand.

Isolated particles can be quantified by counting particles per unit volume, spectrophotometric analysis, lipid mass measurement, SDS-PAGE analysis of polypeptides, or by a functional assay, all of which are easily understood by someone skilled in the science. It is expected that less than about 100 particles will be required to change a phagocyte from characteristics of M1 to characteristics of an M2 and may be as low as 1 particle. Doses given to an organism would be determined by the number of phagocytes desired to be changed and pharmaceutically optimized.

Particles may be administered using a transdermal device, subcutaneously, intravascularly, and through a pharmaceutically acceptable carrier or diluent formulation to an organism where phagocytes bind indirectly through antibodies bound to transmembrane polypeptides LH or directly to complement 3b domains of transmembrane polypeptides T. Subjects administered particles would have all of the benefits afforded by phagocytes having increased catabolism of lipids. Examples of these benefits are not limited to reduction of fatty acids accumulated in the liver due to Hepatitis infections, drug abuse, or hereditary liver disease.

### Example 4

This prospective example demonstrates a kit sold so that the user may deliver polypeptides to cells as needed. The nucleic acids of claim 1 are made available as a kit whereby the end user would ligate a nucleic acid sequence encoding a polypeptide domain of interest into the nucleic acid sequence of the polypeptide to be delivered domain whereby the domain is incorporated into particles. The nucleic acids would be placed into expression vectors so that the nucleic acids of claim 1 are operably linked to an expression control sequence. The kit could include at least 1 transmembrane polypeptide and at least 1 core polypeptide. Nucleic acids encoding core polypeptides comprise sequences encoding the lipid-binding, interaction, and late domains. Nucleic acids encoding core polypeptides comprising a protease domain are also included. Nucleic acids encoding core polypeptides also comprising multi-cloning sites or sites where nucleic acids could be included by someone of ordinary skill in the science. The kit could also include a cell line of claim 3 stably expressing one or more of the nucleic acids of claim 1 operably linked to expression control sequences. The kit could also include a cell line to transfect with the nucleic acids of claim 1 comprised in an expression vector and operably linked to expression control sequences. The kit could include all of the components necessary to generate lipid-enveloped particles comprising mature polypeptides to be delivered.

### How the Invention is Used

In one embodiment of the current invention the nucleic acids are grouped and sold as a kit. The kit includes a cell line of type that is preferred as a producer cell line. The instructions with the kit explain the preferred methods for optimizing the production of particles and how to apply the particles. The kit allows choice of how to use the invention for therapies and experiments.

In another embodiment of the current invention the particles are generated and sold as a pharmaceutical. In this embodiment particles are generated with the desired polypeptide to be delivered domain and the isolated mature particles are included in a pharmaceutically acceptable carrier or diluent formulation.

The amount of particle to be administered and the dosing regimen may vary depending on such factors as desired biological endpoints, routes of administration, target tissues, and severity of the diseases. These factors will be appreciated by those skilled in the science and further developed as production and administration to humans is initiated.

The reaction of the subject's blood to the administered particles should be experimentally determined periodically between and during doses.

### Advantages of The Invention

The present invention described herein has many advantages, including targeting lipid enveloped particles to phagocytes with specificity not previously achieved. A limitation of gene therapy has been neutralization of vectors by a subject's immune system. This immune recognition has also caused painful adverse reactions by the subject, sometimes resulting in death. Unintended effects of gene therapy have been inadvertently amplified by increasing amounts of vector in given doses to increase the likelihood of contacting target cells.

Another advantage of the present invention is a therapy administered in very low doses due to exquisite precision of the immune system in recognition and engulfment of particles and antigens. The present invention uses a transmembrane polypeptide recognized as antigen by phagocytes to mediate this engulfment and a transmembrane polypeptide that fuses lipids of phagocytes with particles. The fusion of lipids results in mixing of contents of particles and phagocytes. The structures of the particles result in a process used as a therapeutic for diseases and as a research product to change the nature of phagocytes, whose role in overcoming disease pathologies is becoming better understood.

Particles are also assembled so that, when desired, nucleic acids are excluded from particles. This provides an advantage in limited “side effects” and unintended, potentially dangerous effects when used in a therapy.

These advantages are not a comprehensive description of all of the advantages of the invention, nor are these advantages required in all versions of the invention.

### Alternatives

Although the present invention has been described in considerable detail with reference to certain preferred versions thereof, other versions are possible. For example, the concepts and functionality of the domains described for core polypeptides and transmembrane polypeptides could be achieved by de novo design of a polypeptide sequence that could not be predicted here. As degeneracy of the genetic code allows multiple nucleic acid triplets to encode single amino acids, multiple amino acids could be linked into variations of polypeptides encoding single domains. Thus, degeneracy also exists in the folds of domains encoded by amino acids. Therefore, the spirit and scope of the appended claims should not be limited to the description of the preferred versions comprised herein.

### REFERENCES

HACEIN-BEY-ABINA S., VON KALLE C., SCHMIDT, M. and LE DEIST, F., (2003) N. Engl. J. Med. 348:255-256.
RAPER S. E., CHIRMULE N., LEE, F. S., WIVEL, N. A., BAGG, A., (2003) Mol. Gen. Metab. 80:148-158.
Guang-ping Gao, b James M. Wilson, b and Mark L. Batshawd,*
KNIPE, HOWLEY, GRIFFIN, LAMB, STRAUS, MARTIN, ROIZMAN. (2007). Fields' Virology. Lippincott, Williams, and Wilkins.
MURPHY, TRAVERS, Walport. (2008). Janeway's Immunobiology Garland Science,
ROGERS G N, PAULSON J C., Virology. (1983)127:361-73.
GLASER L, STEVENS J, ZAMARIN D, WILSON I A, GARCÍA-SASTRE A, TUMPEY T M, BASLER C F, TAUBENBERGER J K, PALESE P., J. Virol. (2005)79:11533-11536.
ZHENG B, CHAN K H, ZHANG A J, ZHOU J, CHAN CC, POON VK, ZHANG K,
LEUNG V H, JIN D Y, WOO P C, CHAN J F, TO K K, CHEN H, YUEN K Y., Exp. Biol. Med. (2010)235:981-988.
PETTIT S C, HENDERSON G J, SCHIFFER C A, SWANSTROM R., J. Virol. (2002)76:10226-10233.
PETTIT S C, LINDQUIST J N, KAPLAN A H, SWANSTROM R., Retrovirology (2005)2:66,
PETTIT S C, CLEMENTE J C, JEUNG J A, DUNN B M, KAPLAN AH., J. Virol. (2005)79:10601.
JOSHI A, MUNSHI U, ABLAN S D, NAGASHIMA K, FREED E O., Traffic (2008)9:1972-1983.
CHUNG H Y, MORITA E, VON SCHWEDLER U, MÜLLER B, KRÄUSSLICH H G, SUNDQUIST W I., J. Virol. (2008)82:4884-4897.
ACCOLA M A, STRACK B, GÖTTLINGER H G., J. Virol. (2000)74:5395-5402.
ZHANG Y, QIAN H, LOVE Z, BARKLIS E., J. Virol. (1998)72:1782-1789.
HARBURY P B, ZHANG T, KIM P S, ALBER T., Science (1993)262:1401-1407.
GOTTWEIN E, BODEM J, MÜLLER B, SCHMECHEL A, ZENTGRAF H, KRÄUSSLICH HG., J. Virol. (2003)77:9474-9485.
ULBRICH P, HAUBOVA S, NERMUT M V, HUNTER E, RUMLOVA M, RUML T., J. Virol. (2006)80:7089-7099.
RUMLOVA-KLIKOVA M, HUNTER E, NERMUT M V, PICHOVA I, RUML T., J. Virol. (2000)74:8452-8459.
BOHMOVÁ K, HADRAVOVÁ R, STOKROVÁ J, TUMA R, RUML T, PICHOVÁ I, RUMLOVÁ M., J. Virol.(2010)84:1977-1988.
LORIAUX M M, REHFUSS R P, BRENNAN R G, GOODMAN R H., PNAS (1993)90:9046-9050.
PARENT L J, BENNETT R P, CRAVEN R C, NELLE T D, KRISHNA N K, BOWZARD J B, WILSON CB, PUFFER B A, MONTELARO R C, WILLS J W., J. Virol. (1995)69:5455-5460.
BORSETTI A, OHAGEN A, GÖTTLINGER H G., J. Virol.(1998)72:9313-9317.
GRIST R M, DATTA S A, STEPHEN A G, SOHEILIAN F, MIRRO J, FISHER R J, NAGASHIMA K, REIN A., J. Virol.(2009)83:2216-2225.
KRÄUSSLICH HG, FÄCKE M, HEUSER A M, KONVALINKAJ, ZENTGRAF H., J. Virol.(1995)69:3407-3419.
ACCOLA M A, HÖGLUND S, GÖTTLINGER H G., J. Virol.(1998)72:2072-2078.
VON SCHWEDLER U K, STEMMLER T L, KLISHKO V Y, LI S, ALBERTINE K H, DAVIS D R, SUNDQUIST W I., EMBO J.(1998)17:1555-1568.
CAMPBELL S, VOGT V M., J. Virol. (1997)71:4425-4435.
JOHNSON M C, SCOBIE H M, M A Y M, VOGT V M., J. Virol. (2002)76:11177-11185.
SHEN B, WU N, YANG J M, GOULD S J., JBC (2011)286:14383-14395.
FANG Y, W U N, GAN X, YAN W, MORRELL J C, GOULD S J., PLOS Biol. (2007)5:e158.
FRANK M. M., AND FRIES L. F., Immunology Today (1991), 12:322-326.
MUDDE G. C., VAN REIJSEN F. C., BOLAND G. J., DE GAST, G. C., BRUIJNZEEL P. L. B., AND BRUIJNZEEL-KOOMEN C. A. F. M., Immunology (1990) 69:335-341
FEIG, J. E., PARATHATH, S, RONG, J. X., MICK, S. L., VENGRENYUK, Y., GRAUER, L, YOUNG, S. G., FISHER, E. A., Circulation (2011) 123:989-998.
PROCHERAY, F., VIAUD, S., RIMANIOL, A.-C., LEONE, C., SAMA H, B., DEREUDDRE-BOSQUET, N., DORMONT, D., GRAS, G., Clin. Exp. Imm. (2005) 142:481-489.
MERKEL, T. J. PERERA, P.-Y., KELLY, V. K., VERMA, A., LLEWELLYN, Z. N., WALDMANN, T. A., MOSCA, J. D., PERERA, L. P., PNAS (2010) 107:18091-18096.
ROITT, BROSTOFF, AND MALE. (1993). Immunology. Mosby publishing.
COFFIN, HUGHES, VARMUS. (1997). Retroviruses. Cold Spring Harbor Press.
SMED-SORENSON, A., CHALOUNI, C., CHATTERJEE, B., COHN, L., BLATTMAN, P., NAKAMURA, N., MELLMAN, I., PloS Pathology (2012). 8(3):e1002572.
STEEL, J., STAEHELI, P., MUBAREKA, S., GARCIA-SASTRE, A., PALESE, P., and LOWEN, A. C., J. Virol. (2010) 84:21-26.

What is claimed is:

1. Isolated nucleic acids comprising sequences encoding polypeptides that assemble to form lipid-enveloped particles that are targeted to the cytoplasm of phagocytes.

2. The nucleic acids of claim 1 operably linked to expression control sequences.

3. A cultured cell comprising the nucleic acids of claim 2.

4. The isolated nucleic acids of claim 1 encoding core polypeptides that assemble to form particles containing polypeptides to be delivered to phagocytes.

5. The isolated nucleic acids of claim 1 comprising sequences encoding polypeptides that assemble to form lipid-enveloped particles, wherein the encoded polypeptides are transmembrane polypeptides.

6. The isolated particles of claim 1 that have been separated from producer cells.

7. A pharmaceutical preparation of the isolated particles of claim 6 wherein the product is useful in therapy or vaccination.

8. (canceled)

9. A kit comprising the nucleic acids of claim 1.
